(19) United States
(12) Patent Application Publication
(10) Pub. No.: US 2023/0398112 A1
(43) Pub. Date: Dec. 14, 2023

(54) SPRAY-DRIED METAL-ORGANIC FRAMEWORKS FOR TREATMENT AND/OR DIAGNOSIS OF PULMONARY DISEASE

(71) Applicant: Research Triangle Institute, Research Triangle Park, NC (US)

(72) Inventors: Ian Edward Stewart, Durham, NC (US); Ignacio Luz Minguez, Durham, NC (US); Anthony James Hickey, Chapel Hill, NC (US)

(21) Appl. No.: 18/020,687

(22) PCT Filed: Jul. 30, 2021
(86) PCT No.: PCT/US2021/043888

§ 371 Date: Feb. 10, 2023

Related U.S. Application Data
(60) Provisional application No. 63/063,785, filed on Aug. 10, 2020.

(51) Int. Cl. A61K 31/4965; A61K 9/16; A61K 9/00; A61K 49/18; A61K 49/04

(52) U.S. Cl. CPC A61K 31/4965; A61K 9/1611; A61K 9/0073; A61K 49/1887; A61K 49/0419

(57) ABSTRACT

Spray-dried metal-organic frameworks (MOFs) comprising therapeutic and/or diagnostic metal ions and therapeutic and/or diagnostic organic ligands are described. Both the metal ion and organic ligand can have activity related to the treatment and/or diagnosis of a pulmonary disease or disorder, such as tuberculosis, or another disease related to a pulmonary infection. The MOFs can be adminstered to subjects via inhalation (e.g., as aerosols). Methods of treating and diagnosing pulmonary diseases or disorders are also described.

### CROSS-REFERENCE TO RELATED APPLICATIONS

This application claims benefit of U.S. Provisional Patent Application Ser. No. 63/063,785, filed Aug. 10, 2020, herein incorporated by reference in its entirety.

### TECHNICAL FIELD

The presently disclosed subject matter relates to spray-dried metal-organic frameworks (MOFs) for the treatment and/or diagnosis of tuberculosis (TB) and other pulmonary diseases or disorders. Both the metal and organic components of the MOFs can have therapeutic activity and/or a property useful for biomedical imaging. The MOFs can be administered via inhalation as a dry powders or suspensions (e.g., using nebulizers or metered dose inhalers), e.g., as aerosols.

### BACKGROUND

More than 10 million people worldwide are infected with tuberculosis (TB) each year, resulting in nearly 1.5 million deaths. [1] Mycobacterium tuberculosis (Mtb) is transmitted by inhalation, followed by alveolar macrophage uptake in the lungs. Current oral and subcutaneous TB regimens can require long treatment duration (≥6 months) and result in systemic side effects that contribute to increased transmission risks and patient compliance issues. In addition, there is an increasing incidence of multi-drug resistant (MDR) and extensively drug resistant (XDR) TB strains. [2]

Accordingly, there is an ongoing need for additional therapeutic compositions and approaches for the treatment of TB, as well as the treatment and/or diagnosis of other pulmonary diseases and disorders.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter provides a spray-dried metal-organic framework (MOF) particle for treating and/or detecting a pulmonary disease or disorder, the spray-dried MOF particle comprising: a metal ion, optionally wherein the metal ion is an ion of an element selected from an alkaline metal, an alkaline earth metal, a transition metal, and a lanthanide; and an organic ligand coordinated to said metal ion, wherein said organic ligand comprises at least two metal coordination sites and wherein the organic ligand has a biological activity related to treatment of the pulmonary disease or disorder, is a prodrug of a therapeutic agent that has a biological activity related to treatment of the pulmonary disease or disorder, and/or has a property useful for biomedical imaging.

In some embodiments, the metal ion has a biological activity related to treatment of the pulmonary disease or disorder and/or a property useful for biomedical imaging, optionally wherein the metal ion is an ion of a transition metal or a lanthanide. In some embodiments, the metal ion is an ion of an element selected from the group comprising copper (Cu), iron (Fe), palladium (Pd), zinc (Zn), silver (Ag), gold (Au), manganese (Mn), cobalt (Co), rhodium (Rh), nickel (Ni), tantalum (Ta), titanium (Ti), tungsten (W), yttrium (Y), vanadium (V), platinum (Pt), gadolinium (Gd), and ytterbium (Yb).

In some embodiments, the organic ligand is a therapeutic agent selected from an anti-asthmatic, an antihistamine, a antitussive, a bronchodilator, a decongestant, an expectorant, a leukotriene modifier, a lung surfactant, an anti-infective, a corticosteroid, a mast cell stabilizer, a mucolytic, a selective phosphodiesterase-4 inhibitor, or a combination or prodrug thereof.

In some embodiments, the spray-dried MOF particle has an average particle diameter of between about 1 μm and about 5 μm, optionally between about 2 μm and about 3 μm. In some embodiments, the spray-dried MOF particle is hollow.

In some embodiments, the organic ligand comprises pyrazinoic acid. In some embodiments, the spray-dried MOF particle comprises bis(pyrazine-2-carboxylato)copper(II) (Cu(POA)₂), optionally wherein said Cu(POA)₂ is hydrated or solvated.

In some embodiments, the spray-dried MOF particle comprises at least two different metal ions. In some embodiments, the spray-dried MOF particle comprises at least two different organic ligands, optionally, wherein each of the at least two different organic ligands are selected from the group comprising an anti-asthmatic, an antihistamine, a antitussive, a bronchodilator, a decongestant, an expectorant, a leukotriene modifier, a lung surfactant, an anti-infective, a corticosteroid, a mast cell stabilizer, a mucolytic, a selective phosphodiesterase-4 inhibitor, or a combination thereof.

In some embodiments, the presently disclosed subject matter provides a therapeutic and/or diagnostic composition, the composition comprising an aerosolized form of a spray-dried MOF particle of the presently disclosed subject matter with an aerodynamic particle size distribution (APSD) in a range suitable for pulmonary delivery, optionally about 1-5 microns. In some embodiments, the composition further comprises an excipient or carrier selected from the group consisting of a sugar, a peptide, a lipid and a surfactant.

In some embodiments, the spray-dried MOF comprises at least one metal ion of an element selected from a transition metal and a lanthanide, optionally at least one metal ion of an element selected from copper (Cu), iron (Fe), palladium (Pd), zinc (Zn), silver (Ag), gold (Au), manganese (Mn), cobalt (Co), rhodium (Rh), nickel (Ni), tantalum (Ta), titanium (Ti), tungsten (W), yttrium (Y), vanadium (V), platinum (Pt), gadolinium (Gd), and ytterbium (Yb). In some embodiments, the spray-dried MOF comprises at least one ligand comprising at least two metal coordination sites and selected from the group comprising an anti-asthmatic, an antihistamine, a antitussive, a bronchodilator, a decongestant, an expectorant, a leukotriene modifier, a lung surfactant, an anti-infective, a corticosteroid, a mast cell stabilizer, a mucolytic, a selective phosphodiesterase-4 inhibitor, or a combination thereof.

In some embodiments, the composition is configured to treat and/or diagnose a pulmonary disease or disorder when administered by inhalation, wherein the pulmonary disease or disorder is selected from a bacterial infection, a viral infection, asthma, chronic obstructive pulmonary disorder (COPD), cystic fibrosis, emphysema, bronchitis, pneumonia, lung cancer and any other disease or disorder treatable using an anti-asthmatic, an antihistamine, an antitussive, a bronchodilator, a decongestant, an expectorant, a leukotriene modifier, a lung surfactant, a corticosteroid, a mast cell stabilizer, a mucolytic, or a selective phosphodiesterase-4 inhibitor. In some embodiments, the therapeutic and/or diagnostic composition is configured to treat tuberculosis (TB) when administered by inhalation.

In some embodiments, the spray-dried MOF particle comprises bis(pyrazine-2-carboxylato)copper(II) (Cu(POA)₂). In some embodiments, the aerosolized formulation of the spray-dried MOF particle comprises an APSD of about 2-3 μm.

In some embodiments, the composition comprises a sugar excipient or carrier, wherein the sugar is selected from maltodextrin, mannitol, trehalose, lactose, and/or dextrose. In some embodiments, the composition comprises a peptide excipient or carrier, wherein said peptide is one or more of leucine, lysine, glycine, polyleucine (dileucine, trileucine), and/or polylysine (dilysine).

In some embodiments, the presently disclosed subject matter provides a method of treating a pulmonary disease or disorder in a subject, the method comprising: providing a subject in need of treatment; providing an inhalable formulation of a spray-dried MOF particle of the presently disclosed subject matter, wherein at least one of the metal ion and the organic ligand has a biological activity related to treatment of the pulmonary disease or disorder; and administering an effective amount of the inhalable formulation of the spray-dried MOF particle to the subject by inhalation, wherein the pulmonary disease or disorder in the subject is treated.

In some embodiments, the inhalable formulation of the spray-dried MOF particle comprises an aerosolized form of the spray-dried MOF particle having an aerodynamic particle size distribution (APSD) in a range suitable for pulmonary delivery, optionally 1-m, further optionally 2-3 μm. In some embodiments, the spray-dried MOF particle is a hollow spray-dried MOF microparticle. In some embodiments, the subject is a human subject.

In some embodiments, the spray-dried MOF comprises a metal ion of an element selected from the group comprising copper (Cu), iron (Fe), palladium (Pd), zinc (Zn), silver (Ag), gold (Au), manganese (Mn), cobalt (Co), rhodium (Rh), nickel (Ni), titanium (Ti), vanadium (V), and platinum (Pt). In some embodiments, the spray-dried MOF comprises an organic ligand having at least two metal coordination sites and which is selected from the group comprising an anti-asthmatic, an antihistamine, an antitussive, a bronchodilator, a decongestant, an expectorant, a leukotriene modifier, a lung surfactant, an anti-infective, a corticosteroid, a mast cell stabilizer, a mucolytic, a selective phosphodiesterase-4 inhibitor, or a combination or prodrug thereof.

In some embodiments, the subject has a pulmonary disease or disorder selected from a bacterial infection, a viral infection, asthma, chronic obstructive pulmonary disorder (COPD), cystic fibrosis, emphysema, bronchitis, pneumonia, lung cancer, and any other disease or disorder treatable using an anti-asthmatic, an antihistamine, an antitussive, a bronchodilator, a decongestant, an expectorant, a leukotriene modifier, a lung surfactant, a corticosteroid, a mast cell stabilizer, a mucolytic, or a selective phosphodiesterase-4 inhibitor. In some embodiments, the subject has tuberculosis (TB). In some embodiments, the TB in the subject comprises multiple drug resistant (MDR) TB or extensively drug resistant (XDR) TB.

In some embodiments, the spray-dried MOF particle comprises bis(pyrazine-2-carboxylato)copper(II) (Cu(POA)₂). In some embodiments, the spray-dried MOF particle further comprises a metal ion selected from Fe, Pd, Zn and Gd.

In some embodiments, the subject is also treated with a second therapeutic composition for treating the pulmonary disease or disorder, wherein the second therapeutic composition is administered orally or intravenously.

In some embodiments, the presently disclosed subject matter provides a method of diagnosing a pulmonary disease or disorder in a subject, the method comprising: providing a subject suspected or at risk of having a pulmonary disease or disorder; providing an inhalable formulation of a spray-dried MOF particle of the presently disclosed subject matter, wherein the spray-dried MOF comprises at least one metal ion or at least one organic ligand having a property useful in biomedical imaging; administering an effective amount of the inhalable formulation of the spray-dried MOF particle to the subject by inhalation; imaging at least a portion of at least one lung of the subject; and analyzing results of the imaging and finding evidence of the presence of a pulmonary disease or disorder; thereby diagnosing the pulmonary disease or disorder in the subject. In some embodiments, the spray-dried MOF particle comprises a metal ion of an element selected from Gd, Fe, Mn, Au, Y, Yb, Ni, Cu, W, Ta, Pt, and Ti, optionally wherein the metal ion is an Gd ion, a Fe ion, or a Mn ion. In some embodiments, the imaging comprises performing magnetic resonance imaging (MRI) or computed tomography (CT).

Accordingly, it is an object of the presently disclosed subject matter to provide spray-dried MOFs for the treatment and/or diagnosis of pulmonary disease, such as TB, therapeutic and/or diagnostic (e.g., theragnostic) compositions comprising the MOFs, and methods of treating and diagnosing pulmonary disease using the MOFs.

Certain objects of the presently disclosed subject matter having been stated hereinabove, which are addressed in whole or in part by the presently disclosed subject matter, other objects and aspects will become evident as the description proceeds when taken in connection with the accompanying Examples as best described herein below.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the below drawings.

FIG. 1A is a schematic drawing showing the reversible hydration and dehydration of a copper-pyrazinoate (Cu(POA)₂ μmetal-organic framework (MOF) (bottom) and a hydrated copper-pyrazinoate (Cu(POA)₂·2H₂O) MOF (top).

FIG. 1B is a graph showing the thermogravimetric analysis (TGA) of pyrazinoic acid (HPOA), hydrated copper-pyrazinoate (Cu(POA)₂·2H₂O), and dehydrated copper-pyrazinoate (Cu(POA)₂).

FIG. 1C is a graph showing the X-ray powder diffraction (XRPD) patterns of hydrated copper-pyrazinoate (Cu(POA)₂·2H₂O) and dehydrated copper-pyrazinoate (Cu(POA)₂).

FIG. 1D is a graph showing the Fourier transform infrared (FTIR) spectra of hydrated copper-pyrazinoate (Cu(POA)₂·2H₂O) and dehydrated copper-pyrazinoate (Cu(POA)₂).

FIG. 2A is a graph showing the histogram of the particle size (in microns (m) of copper-pyrazoinoate (Cu(POA)₂) aerosol particles obtained using spray-drying.

FIG. 2B is a scanning electron microscopy (SEM) image of copper-pyrazinoate (Cu(POA)₂) aerosol particles obtained by spray-drying.

FIG. 2C is a transmission electron microscopy (TEM) image of copper-pyrazinoate (Cu(POA)₂) aerosol particles obtained by spray-drying.

FIG. 3A is a graph showing the inertial impactor data for copper pyrazinoate (Cu(POA)₂) aerosol particles obtained by spray-drying.

FIG. 3B is a graph showing the dry powder delivery data measured from a custom dosator for copper-pyrazinoate (Cu(POA)₂) aerosol particles obtained under the optimal spray dryer synthesis parameters.

FIG. 4 is a series of microscope images including (left) an optical microscopy image of bulk copper-pyrazinoate (Cu(POA)₂) metal-organic framework (MOF) particles prepared at room temperature; (second from left) a scanning electron microscopy (SEM) image of bulk Cu(POA)₂ MOF particles prepared at room temperature; (second from right) a SEM image of bulk Cu(POA)₂ MOF particles prepared at 60 degrees Celsius (° C.); and (right) a SEM image of hollow spherical Cu(POA)₂ MOF particles manufactured via spray drying using a 150° C. inlet temperature.

FIG. 5 is a graph showing the X-ray powder diffraction patterns of spray-dried copper-pyrazinoate metal-organic framework materials prepared under different conditions: 150 degrees Celsius (° C.) inlet temperature, 1 copper (Cu): 1.6 pyrazinoic acid (POA), 1.5 mg pyrazinoic acid per milliliter (POA/mL), 100% water (H₂O) and 293 liters per hour (L/h) nitrogen (N2) flow (15 kilopascal (kPa) pressure drop) (top, Condition C); 150° C. inlet temperature, 1 Cu: 1.6 POA, 1.5 mg POA/mL, 80%:20% H₂O:ethanol (EtOH) and 293 L/h N2 flow (15 kPa pressure drop) (second from top, Condition B); and 180° C. inlet temperature, 1 Cu: 2 POA, 1.5 mg POA/mL, 80%:20% H₂O:EtOH and 293 L/h N2 flow (15 kPa pressure drop) (third from top, Condition A). These patterns are compared to bulk hydrated copper-pyrazinoate (Cu(POA)₂ H₂O batch); bulk dehydrated copper-pyrazinoate (Cu(POA)₂ batch), a simulated PXRD pattern for dehydrated Cu(POA)₂ (Cu(POA)₂ reference), and pyrazinoic acid (H-POA).

FIG. 6 is a graph showing the Fourier transform infrared (FTIR) analysis for spray dried copper-pyrazinoate metal-organic framework materials prepared under the following conditions: 150 degrees Celsius (° C.) inlet temperature, 1 copper (Cu): 1.6 pyrazinoic acid (POA), 1.5 mg pyrazinoic acid per milliliter (POA/mL), 100% water (H₂O) and 293 liters per hour (L/h) nitrogen (N2) flow (15 kilopascals (kPa) pressure drop) (Condition A); 150° C. inlet temperature, 1 Cu: 1.6 POA, 1.5 mg POA/mL, 80%:20% H₂O:ethanol (EtOH) and 293 L/h N2 flow (15 kPa pressure drop) (Condition B); and 180° C. inlet temperature, 1 Cu: 2 POA, 1.5 mg POA/mL, 80%:20% H₂O:EtOH and 293 L/h N2 flow (15 kPa pressure drop) (Condition A). The spectra are compared to bulk dehydrated copper-pyrazinoate (Cu(POA)₂) (top) and pyrazinoic acid (POA, bottom).

FIG. 7A is a pair of scanning electron microscopy (SEM) images of spray dried copper-pyrazinoate (Cu(POA)₂) metal-organic framework (MOF) materials prepared using an inlet temperature of (left top) 150 degrees Celsius (° C.) or (left bottom) 180° C. FIG. 7B is a graph of the X-ray powder diffraction patterns of Cu(POA)₂ MOF materials spray dried using an inlet temperature of 150° C. (middle line) or 180° C. (top line) compared to bulk dehydrated Cu(POA)₂ (bottom line); and (right) a graph showing the Fourier transform infrared (FTIR) spectra for pyrazinoic acid (HPOA). FIG. 7C shows the results of Cu(POA)₂ MOF materials spray dried using an inlet temperature of 150° C. (second from top spectrum) or 180° C. (second from bottom spectrum) compared to bulk dehydrated Cu(POA)₂ (top spectrum) and pyrazinoic acid (HPOA, bottom spectrum). The other spray drying input parameters that were held constant during this temperature variation were: 1 copper (Cu): 2 pyrazinoic acid (POA), 1.5 mg POA/milliliter (mL), 80%:20% H₂O:EtOH and 293 liters per hour (L/h) nitrogen (N2) flow (15 kilopascal (kPa) pressure drop).

FIGS. 8A-8C show graphs of the laser diffraction data (counts (volume %)) versus particle size (microns (m)) for copper-pyrazinoate (Cu(POA)₂) metal-organic framework microparticles evaluating the effect of precursor concentration on volume particle size. For FIG. 8A, the ratio of copper (Cu) to pyrazinoic acid (POA) was 1:2 at 1.0 milligrams per milliliter (mg/mL) POA. For FIG. 8B, the ratio of Cu to POA was 1:2 at 1.5 mg/mL POA. For FIG. 8C, the ratio of Cu to POA was 1:2 at 3.0 mg/mL POA. The other spray drying input parameters that were held constant during this concentration variation were: 180 degrees Celsius (° C.) inlet temperature; 80% water (H₂O) to 20% ethanol (EtOH); and 293 liters per hour (L/h) nitrogen (N2) flow (15 kilopascal (kPa) pressure drop).

FIGS. 9A and 9B show graphs of the laser diffraction data (counts (volume %)) versus particle size (microns (m)) of copper-pyrazinoate (Cu(POA)₂) metal-organic framework (MOF) microparticles evaluating the effect of atomizing gas flow (nitrogen (N2)) on volume particle size. FIG. 9A shows the effect of low N2 flow, i.e., at 293 liters per hour (L/h) (15 kilopascal (kPa) pressure drop). FIG. 9B shows the effect of high N2 flow, i.e., at 1052 L/h (75 kPa pressure drop). The other spray drying input parameters that were held constant during this variation were 1:2 copper: pyrazinoic acid, 150 degrees Celsius inlet temperature, 1.5 milligrams per milliliter (mg/mL) pyrazinoic acid, and 80% water:20% ethanol.

FIG. 10 is a series of scanning electron microscopy (SEM) images for evaluating the effect of precursor concentration on the spray dried metal-organic framework (MOF) materials (dN=nanoparticle diameter; dP=total particle diameter). Spray drying input parameters that were held constant during this concentration variation were: 1 copper (Cu): 2 pyrazinoic acid (POA), 180 degrees Celsius inlet temperature, 80%:20% water:ethanol, and 293 liters per hour (L/h) nitrogen (N2) flow (15 kilopascal (kPa) pressure drop).

FIGS. 11A-11B show a pair of scanning electron microscopy (SEM) images for evaluating the effect of the nitrogen (N2) flow on the spray dried metal-organic framework (MOF) materials. Low N2 (FIG. 11A)=293 liters per hour (L/h) flow rate (15 kilopascal (kPa) pressure drop) and high N2 (FIG. 11B)=1052 L/h (75 kPa pressure drop). Spray drying input parameters that were held constant during this atomizing gas variation were: 1 copper (Cu): 2 pyrazinoic acid (POA), 150 degrees Celsius inlet temperature, 1.5 mg POA per milliliter and 80%:20% water:ethanol. To the right of the SEM images are corresponding particle size histograms.

FIG. 12 is a series of scanning electron microscopy (SEM) images for (left) copper-pyrazinoate (Cu(POA)₂) meta-organic framework (MOF) particles obtained under the following spray drying conditions: 1:2 copper:pyrazinoic acid; 15 milligrams pyrazinoate per milliliter; 180 degrees Celsius inlet temperature; 80%:20% water:ethanol; and 293 liters per hour nitrogen flow (15 kilopascal pressure drop); and Cu(POA)₂ after soaking/sonicating in methanol (top right) and water (bottom left).

FIG. 13A is a graph of Next Generation Impactor (NGI) data for copper-pyrazinoate (Cu(POA)₂) metal-organic framework (MOF) particles spray dried using an inlet temperature of 180 degrees Celsius (° C.) and low nitrogen flow (293 liters per hour (L/h) (15 kilopascal (kPa) pressure drop)). A 1:2 molar ratio of copper to pyrazinoic acid was used. The open bars show the mass data for copper (Cu) and the filled bars show the mass data for pyrazinoic acid (POA).

FIG. 13B is a graph of Next Generation Impactor (NGI) data for copper-pyrazinoate (Cu(POA)₂) metal-organic framework (MOF) particles spray dried using an inlet temperature of 150 degrees Celsius (° C.) and low nitrogen flow (293 liters per hour (L/h) (15 kilopascal (kPa) pressure drop)). A 1:2 molar ratio of copper to pyrazinoic acid was used. The open bars show the mass data for copper (Cu) and the filled bars show the mass data for pyrazinoic acid (POA).

FIG. 14 is (left) a scanning electron microscopy (SEM) image and (right) electron dispersive spectroscopy (EDS) analysis for an exemplary theragnostic material of the presently disclosed subject matter Gd_{0.1}Cu_{0.9}(POA)₂, showing the presence of both gadolinium (Gd) and copper (Cu) in a single spherical particle. Gd_{0.1}Cu_{0.9}(POA)₂ microparticles were spray dried under the same conditions as the copper-pyrazinoate microparticles described in FIG. 3A but with a metal precursor molar concentration ratio of 1:9 Gd:Cu (using (Gd(NO₃)₃ salt.

### DETAILED DESCRIPTION

The presently disclosed subject matter will now be described more fully hereinafter with reference to the accompanying Figures and Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist, unless as otherwise specifically indicated.

### I. Definitions

Following long-standing patent law convention, the terms “a”, “an”, and “the” refer to “one or more” when used in this application, including the claims. Thus, for example, reference to “a solvent” includes mixtures of one or more solvents, two or more solvents, and the like.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the presently disclosed subject matter.

The term “about”, as used herein when referring to a measurable value such as an amount of weight, molar equivalents, time, temperature, etc. is meant to encompass in one example variations of ±20% or ±10%, in another example ±5%, in another example ±1%, and in yet another example ±0.1% from the specified amount, as such variations are appropriate to perform the disclosed methods.

The term “and/or” when used to describe two or more activities, conditions, or outcomes refers to situations wherein both of the listed conditions are included or wherein only one of the two listed conditions are included.

The term “comprising”, which is synonymous with “including,” “containing,” or “characterized by” is inclusive or open-ended and does not exclude additional, unrecited elements or method steps. “Comprising” is a term of art used in claim language, which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein the term “alkyl” refers to C1-C20 inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C1-C8 alkyl), e.g., 1, 2, 3, 4, 5, 6, 7, or 8 carbon atoms. In some embodiments, “lower alkyl” can refer to C1-C6 or C1-C5 alkyl groups. “Higher alkyl” refers to an alkyl group having about 10 to about carbon atoms, e.g., 10, 11, 12, 13, 14, 15, 16, 17, 18, 19, or 20 carbon atoms. In certain embodiments, “alkyl” refers, in particular, to C1-C8 or C1-C6 straight-chain or branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, nitro, cyano, amino, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, cyano, amino, alkylamino, dialkylamino, ester, acyl, amide, sulfonyl, sulfate, and mercapto.

The term “alkenyl” refers to an alkyl group as defined above including at least one carbon-carbon double bond. Exemplary alkenyl groups include, but are not limited to, ethenyl, propenyl, butenyl, pentenyl, hexenyl, octenyl, butadienyl, and allenyl groups.

Alkenyl groups can optionally be substituted with one or more alkyl group substitutents, which can be the same or different, including, but not limited to alkyl (saturated or unsaturated), substituted alkyl (e.g., halo-substituted and perhalo-substituted alkyl, such as but not limited to, —CF₃), cycloalkyl, halo, nitro, hydroxyl, carbonyl, carboxyl, acyl, alkoxyl, aryloxyl, aralkoxyl, thioalkyl, thioaryl, thioaralkyl, amino (e.g., aminoalkyl, aminodialkyl, aminoaryl, etc.), sulfonyl, and sulfinyl.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. In some embodiments, the cycloalkyl ring system comprises between 3 and 6 carbon atoms. The cycloalkyl group can be optionally partially unsaturated. The cycloalkyl group also can be optionally substituted with an alkyl group substituent as defined herein. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include, but are not limited to, cyclopropyl, cyclobutyl, cyclopentyl, cyclohexyl, cycloheptyl, and the like. Further, the cycloalkyl group can be optionally substituted with a linking group, such as an alkylene group as defined hereinbelow, for example, methylene, ethylene, propylene, and the like. In such cases, the cycloalkyl group can be referred to as, for example, cyclopropylmethyl, cyclobutylmethyl, and the like. Additionally, multicyclic cycloalkyl rings include adamantyl, octahydronaphthyl, decalin, camphor, camphane, and noradamantyl.

Thus, as used herein, the term “substituted cycloalkyl” includes cycloalkyl groups, as defined herein, in which one or more atoms or functional groups of the cycloalkyl group are replaced with another atom or functional group, including for example, alkyl, substituted alkyl, halogen, aryl, substituted aryl, alkoxyl, hydroxyl, nitro, cyano, amino, alkylamino, dialkylamino, ester, acyl, amide, sulfonyl, sulfate, and mercapto.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds (i.e., “heteroaryl”). The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyridine, imidazole, benzimidazole, isothiazole, isoxazole, pyrazole, pyrazine, triazine, thiazole, pyrimidine, quinoline, isoquinoline, indole, carbazole, napthyl, and the like.

“Heterocyclic”, “heterocycle”, or “heterocyclo” as used herein alone or as part of another group, refers to an aliphatic (e.g., fully or partially saturated heterocyclo) or aromatic (e.g., heteroaryl) monocyclic- or a bicyclic-ring system comprising one or more heteroatoms (e.g., 1, 2, or 3 heteroatoms selected from oxygen, sulfur, and substituted or unsubstituted nitrogen) inserted along the cyclic alkyl or aryl carbon chain. Monocyclic ring systems are exemplified by any 5- or 6-membered ring containing 1, 2, 3, or 4 heteroatoms independently selected from oxygen, nitrogen and sulfur. The 5 membered ring has from 0-2 double bonds and the 6 membered ring has from 0-3 double bonds. Representative examples of monocyclic ring systems include, but are not limited to, ethylene oxide, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydropyran, tetrahydrothiophene (also known as thiolane), tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein.

Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, carbazole, cinnoline, indazole, indole, indoline, indolizine, naphthyridine, isobenzofuran, isobenzothiophene, isoindole, isoindoline, isoquinoline, phthalazine, purine, pyranopyridine, quinoline, quinolizine, quinoxaline, quinazoline, tetrahydroisoquinoline, tetrahydroquinoline, thiopyranopyridine, and the like. These rings include quaternized derivatives thereof and can be optionally substituted with one or more alkyl and/or aryl group substituents.

“Substituted heterocyclic” as used herein refers to a heterocyclic group wherein one or more hydrogen atom is replaced by an alkyl or aryl group substitutent.

The term “N-heterocycle” refers to a heterocycle wherein at least one of the heteroatoms is a nitrogen atom. Examples of N-heterocycles include, but are not limited to, azetidine, pyrrolidine, pyrrole, pyrroline, pyrazole, pyrazoline, pyrazolidine, piperidine, pyridine, piperazine, pyrazine, pyrimidine, pyridazine, morpholine, imidazole, benzimidazole, imidazoline, imidazolidine, indole, carbazole, quinoline, isoquinoline, oxazole, thiazole, isothiazole, and thiazine.

“Substituted N-heterocycle” refers to a N-heterocycle wherein one or more hydrogen is replaced by an alkyl or aryl group substituent.

The term “heteroaryl” referes to an aromatic monocyclic- or a bicyclic-ring system (a fused, bridged or spirocyclic ring system) comprising one or more heteroatoms (e.g., 1, 2, or 3 heteroatoms selected from oxygen, sulfur, and substituted or unsubstituted nitrogen, wherein N-oxides, sulfur oxides and dioxides are permissible heteroatom substitutions) inserted along the cyclic aryl carbon chain. In some embodiments, the monocyclic heteroaryl group is a five to seven membered aromatic ring. Representative heteroaryl groups include, but are not limited to, furan, thiophene, pyrrole, imidazole, pyrazole, triazole, tetrazole, oxazole, isoxazole, oxadiazole, thiaciazole, isothiazole, pyridine, pyridazine, pyrazine, pyrimidine, quinoline, isoquinoline, benzofuran, benzoxazole, benzothiophene, indole, indazole, benzimidazole, imidazopyridine, pyrazolopyrindine, and pyrazolopyrimidine.

The term “substituted heteroaryl” refers to a heteroaryl group as defined herein wherein one or more hydrogen atoms is replaced by an aryl group substituent.

“Aralkyl” refers to an aryl-alkyl- or an -alkyl-aryl group wherein aryl and alkyl are as previously described and can include substituted aryl and substituted alkyl. Thus, “substituted aralkyl” can refer to an aralkyl group comprising one or more alkyl or aryl group substituents. Exemplary aralkyl groups include benzyl, phenylethyl, and naphthylmethyl.

“Alkylene” can refer to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated (i.e., include alkene or alkyne groups) and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

“Arylene” refers to a bivalent aryl group, which can be substituted or unsubstituted.

The term “aralkylene” refers to a bivalent group that comprises a combination of alkylene and arylene groups (e.g., -arylene-alkylene-, alkylene-arylene-alkylene-, arylene-alkylene-arylene-, etc.).

Similarly, the terms “cycloalkylene”, “heterocycloalkylene” and “heteroarylene” refer to bivalent cycloalkyl, heterocyclic, and heteroaryl groups, which can optionally be substituted with one or more alkyl or aryl group substitutents.

As used herein, the term “acyl” refers to an organic carboxylic acid group wherein the —OH of the carboxylic acid group has been replaced with another substituent. Thus, an acyl group can be represented by RC(═O)—, wherein R is an alkyl, substituted alkyl, aralkyl, substituted aralkyl, aryl or substituted aryl group as defined herein. As such, the term “acyl” specifically includes arylacyl groups, such as a phenacyl group. Specific examples of acyl groups include acetyl (i.e., —C(═O)CH₃) and benzoyl.

“Alkoxyl” refers to an alkyl-O— group wherein alkyl is as previously described, including substituted alkyl. The term “alkoxyl” as used herein can refer to, for example, methoxyl, ethoxyl, propoxyl, isopropoxyl, butoxyl, t-butoxyl, and pentoxyl. The terms “oxyalkyl” and “alkoxy” can be used interchangeably with “alkoxyl”.

“Aryloxyl” and “aryloxy” refer to an aryl-O— group wherein the aryl group is as previously described, including a substituted aryl. The term “aryloxyl” as used herein can refer to phenyloxyl or hexyloxyl, and to alkyl, substituted alkyl, or alkoxyl substituted phenyloxyl or hexyloxyl.

“Aralkyloxyl” or “aralkoxy” refer to an aralkyl-O— group wherein the aralkyl group is as previously described. An exemplary aralkyloxyl group is benzyloxyl.

The term “carbonyl” refers to the group —C(═O)—. The term “carbonyl carbon” refers to a carbon atom of a carbonyl group. Other groups such as, but not limited to, acyl groups, anhydrides, aldehydes, esters, lactones, amides, ketones, carbonates, and carboxylic acids, include a carbonyl group.

The terms “carboxyl” and “carboxylic acid” refer to the —C(═O)OH or —C(═O)O⁻ group. In some embodiments, the term “carboxylate” refers to the C(═O)O⁻ group.

The term “acid chloride” can refer to the —C(═O)Cl group.

The terms “halo” or “halogen” as used herein refer to fluoro, chloro, bromo, and iodo groups.

The term “haloalkyl” refers to an alkyl group as defined herein substituted by one or more halo groups.

The term “perhaloalkyl” refers to an alkyl group as defined herein wherein all C—H bonds are replaced by carbon-halogen bonds. The term “perfluoroalkyl” refers to an alkyl group wherein all C—H bonds are replaced by C—F bonds. An exemplary perfluoroalkyl group is trifluoromethyl (—CF₃).

The term “sulfonyl” refers to the —S(═O)₂R group, wherein R is alkyl, substituted alkyl, aralkyl, substituted aralkyl, aryl, or substituted aryl. The term “alkylsulfonyl” refers to the —S(═O)₂R group, wherein R is alkyl or substituted alkyl. In some embodiments, the sulfonyl group is —S(═O)₂CH₃.

The term “ester” refers to the R′—O—C(═O)— group, wherein the carbonyl carbon is attached to another carbon atom and wherein R′ is alkyl, cycloalkyl, aralkyl, or aryl, wherein the alkyl, cycloalkyl, aralkyl, or aryl are optionally substituted. The term “esterifying” can refer to forming an ester by contacting a compound containing a carboxylic acid or derivative thereof (e.g., an acid chloride) and a compound containing a hydroxyl group (e.g., an alcohol or a phenol).

The term “amide” refers to a compound comprising the structure R′—NR″—C(═O)—R, wherein R is alkyl, substituted alkyl, aralkyl, substituted aralkyl, aryl or substituted aryl, and wherein R′ and R″ are independently hydrogen, alkyl, aralkyl, or aryl, wherein the alkyl, aralkyl, or aryl are optionally substituted. In some embodiments, R′ is alkyl, substituted alkyl, aralkyl, substituted aralkyl, aryl, or substituted aryl.

The term “amine” refers to a molecule having the formula N(R)₃, or a protonated form thereof, wherein each R is independently H, alkyl, substituted alkyl, aryl, substituted aryl, aralkyl, substituted aralkyl, or wherein two R groups together form an alkylene or arylene group. The term “primary amine” refers to an amine wherein at least two R groups are H. The term “secondary amine” refers to an amine wherein only one R group is H. The term “alkylamine” can refer to an amine wherein two R groups are H and the other R group is alkyl or substituted alkyl. “Dialkylamine” can refer to an amine where two R groups are alkyl. “Arylamine” can refer to an amine wherein one R group is aryl. Amines can also be protonated, i.e., have the formula [NH(R)₃]⁺.

The term “amino” refers to the —N(R)₂ group wherein each R is independently H, alkyl, substituted alkyl, aryl, substituted aryl, aralkyl, or substituted aralkyl. The terms “aminoalkyl” and “alkylamino” can refer to the —N(R)₂ group wherein each R is H, alkyl or substituted alkyl, and wherein at least one R is alkyl or substituted alkyl. The term “dialkylamino” refers to an aminoalkyl group where both R groups are alkyl or substituted alkyl, which can be the same or different.

The terms “acylamino” and “aminoacyl” refer to the —N(R)—C(═O)R′ group, wherein R is selected from H, alkyl, substituted alkyl, aralkyl, substituted aralkyl, aryl, and substituted aryl, and wherein R′ is selected from alkyl, substituted alkyl, aralkyl, substituted aralkyl, aryl, and substituted aryl.

The term “cyano” refers to the —C≡N group.

The terms “hydroxyl” and “hydroxy” refer to the —OH group.

The terms “mercapto” and “thiol” refer to the —SH group.

The term “oxo” refers to a compound described previously herein wherein a carbon atom is replaced by an oxygen atom.

The term “nitro” refers to the —NO₂ group.

The term “thioalkyl” can refer to the group —SR, wherein R is selected from H, alkyl, substituted alkyl, aralkyl, substituted aralkyl, aryl, and substituted aryl. Similarly, the terms “thioaralkyl” and “thioaryl” refer to —SR groups wherein R is aralkyl and aryl, respectively.

The terms “bonding” or “bonded” and variations thereof can refer to either covalent or non-covalent bonding. In some cases, the term “bonding” refers to bonding via a coordinate bond. The term “conjugation” can refer to a bonding process, as well, such as the formation of a covalent linkage or a coordinate bond.

As used herein, the term “metal-organic framework” refers to a solid one-, two-, or three-dimensional network comprising both metal and organic components, wherein the organic components include at least one, and typically more than one carbon atom. In some embodiments, the material is crystalline. In some embodiments, the material is amorphous. In some embodiments, the material is porous. In some embodiments, the metal-organic matrix material is a coordination polymer, which comprises repeating units of a coordination complex comprising a metal-based component, e.g., a metal ion, a metal cluster, or a metal-oxo cluster, and an organic ligand (e.g., a bidentate, tridentate, or other polydentate organic ligand). In some embodiments, the material contains more than one type of metal-based component. In some embodiments, the material can contain more than one type of organic bridging ligand.

A “coordination complex” is a compound in which there is a coordination bond between a metal ion and an electron pair donor, ligand or chelating group. Thus, ligands or chelating groups are generally electron pair donors, molecules or molecular ions having unshared electron pairs available for donation to a metal ion.

The term “coordination bond” refers to an interaction between an electron pair donor and a coordination site on a metal ion resulting in an attractive force between the electron pair donor and the metal ion. The use of this term is not intended to be limiting, in so much as certain coordinate bonds also can be classified as having more or less covalent character (if not entirely covalent character) depending on the characteristics of the metal ion and the electron pair donor.

As used herein, the term “ligand” refers generally to a species, such as a molecule or ion, which interacts, e.g., binds, in some way with another species. More particularly, as used herein, a “ligand” can refer to a molecule or ion that binds a metal ion in solution to form a “coordination complex.” See Martell, A. E., and Hancock, R. D., Metal Complexes in Aqueous Solutions, Plenum: New York (1996), which is incorporated herein by reference in its entirety. The terms “ligand” and “chelating group” can be used interchangeably. The term “bridging ligand” can refer to a group that bonds to more than one metal ion or complex, thus providing a “bridge” between the metal ions or complexes. Organic bridging ligands can have two or more groups with unshared electron pairs separated by, for example, an alkylene or arylene group. Groups with unshared electron pairs, include, but are not limited to, —CO₂H, —NO₂, amino, hydroxyl, thio, thioalkyl, —B(OH)₂, —SO₃H, PO₃H, phosphonate, and heteroatoms (e.g., nitrogen, oxygen, or sulfur) in heterocycles.

The term “coordination site” when used herein with regard to a ligand, e.g., a bridging ligand, refers to a unshared electron pair, a negative charge, or atoms or functional groups cable of forming an unshared electron pair or negative charge (e.g., via deprotonation under at a particular pH).

The terms “microscale particle” and “microparticle” refer to a structure having at least one region with a dimension (e.g., length, width, diameter, etc.) of less than about 1,000 microns (m). In some embodiments, the dimension is smaller (e.g., less than about 500 μm), less than about 250 μm, less than about 200 μm, less than about 150 μm, less than about 125 μm, less than about 100 μm, less than about 80 μm, less than about 70 m, less than about 60 μm, less than about 50 μm, less than about 40 μm, less than about m or even less than about 20 μm). In some embodiments, the dimension is between about 0.1 μm and about 15 μm (e.g., about 0.1, 0.5, 1, 2, 3, 4, 5, 6, 7, 8, 9, 10, 11, 12, 13, 14, or about 15 μm). In some embodiments, the dimension is between about 0.1 μm and about 5 μm (e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0 μm).

In some embodiments, the microparticle is approximately spherical. When the microparticle is approximately spherical, the characteristic dimension can correspond to the diameter of the sphere. In addition to spherical shapes, the microparticle can be disc-shaped, plate-shaped (e.g., hexagonally plate-like), oblong, polyhedral, rod-shaped, cubic, or irregularly-shaped.

The microparticle can comprise a core region (i.e., the space between the outer dimensions of the particle) and an outer surface (i.e., the surface that defines the outer dimensions of the particle). In some embodiments, the microparticle can have one or more coating layers surrounding or partially surrounding the microparticle core. Thus, for example, a spherical microparticle can have one or more concentric coating layers, each successive layer being dispersed over the outer surface of a smaller layer closer to the center of the particle.

In some embodiments, the presently disclosed microparticles can comprise a solid metal-organic framework (MOF) matrix, which are two- or three-dimensional networks of metal-organic ligand coordination complexes. The MOF matrix can be amorphous or crystalline. In some embodiments, the MOF particles are hollow.

“Embedded” can refer to an agent that is bound, for example covalently bound or bound via a coordinative bond, inside the core of a microparticle (e.g., to a coordination site of a organic bridging ligand or to a metal ion). Alternatively, agents can be “sequestered”, “entrapped”, or “trapped” (i.e., non-covalently encapsulated) inside pores, cavities or channels in the core of an MOF particle or interact with a MOF material via hydrogen bonding, London dispersion forces, or any other non-covalent interaction.

The terms “polymer” and “polymeric” refer to chemical structures that have repeating units (i.e., multiple copies of a given chemical substructure). Polymers can be formed from polymerizable monomers. A polymerizable monomer is a molecule or complex that comprises one or more moieties that can react or interact to form bonds (e.g., covalent or coordination bonds) with moieties on other molecules or complexes of polymerizable monomer. In some embodiments, each polymerizable monomer can bond to two or more other molecules/moieties. In some cases, a polymerizable monomer will bond to only one other molecule, forming a terminus of the polymeric material.

Polymers can be organic, or inorganic, or a combination thereof. As used herein, the term “inorganic” refers to a compound or composition that contains at least some atoms other than carbon, hydrogen, nitrogen, oxygen, sulfur, phosphorous, or one of the halides. Thus, for example, an inorganic compound or composition can contain one or more silicon atoms and/or one or more metal atoms.

The terms “treatment” and “treating” and the like as used herein refers to any treatment of a disease and/or condition in an animal or mammal, particularly a human, and includes: (i) preventing a disease, disorder and/or condition from occurring in a person which can be predisposed to the disease, disorder and/or condition, or at risk for being exposed to an agent that can cause the disease, disorder, and/or condition; but, has not yet been diagnosed as having it; (ii) inhibiting the disease, disorder and/or condition, i.e., arresting its development; and (iii) relieving the disease, disorder and/or condition, i.e., causing regression of the disease, disorder and/or condition.

The terms “biomedical imaging” or “imaging” refer to techniques known in the field of medical and/or veterinary diagnosis, such as magnetic resonance imaging (MRI), computed x-ray tomography (CT or X-ray CT), optical imaging (OI), positron emission tomography (PET), and single-photon emission computed tomography (SPECT).

### II. General Considerations

Pulmonary delivery is a promising alternative strategy to combat TB and other pulmonary diseases or disorders as compared to oral, intravenous, or subcutaneous delivery of therapeutic agents. For example, with regard to TB, targeting the lungs offers an inherent advantage of direct access of the drug to the primary site of Mtb infection thus maximizing drug delivery (>minimum inhibitory concentration (MIC)) while avoiding toxic systemic side effects, potentially shortening treatment duration.[3] Indeed inhaled therapies for TB are gaining attention, especially the use of dry powder inhalers (DPIs).[3,4,5] In particular, developing countries, where most TB deaths occur, can benefit from these easy-to-use, disposable, and compact devices, that do not require refrigeration, electricity, or needles. Non-traditional methods of drug delivery, like pulmonary delivery, can also play an important role in anti-TB treatment by overcoming resistance to current treatment options.

Until new anti-TB regimens are established, one of the first-line drugs considered indispensable in avoiding efficacy loss is pyrazinamide (PZA). [6] Briefly, the mechanism of action of PZA is to preferentially penetrate hypoxic and acidic granulomas where macrophages containing persistent Mtb are sequestered.[7] It is then converted to the active moiety, pyrazinoic acid (POA), and action toward non-replicating Mtb within the granulomas can occur.[8] Despite PZA being an effective component of TB therapy, almost 20% of TB and more than 50% of MDR TB cases have developed resistance to PZA by preventing Mtb conversion of PZA to POA.[9] Consequently, POA itself has been explored as an alternative form of TB therapy to bypass PZA resistance but has proven ineffective when delivered orally. [10] When delivered as an aerosol, however, POA esters (PAEs; prodrugs for POA [11]) helped reduce the burden of Mtb in the lung and spleen compared with untreated animals.[12] Spray dried powders of POA salts [13], and POA+PAEs [14], have also been manufactured for anti-TB potential and the latter demonstrated efficacy toward Mtb in animal models, but much is still yet to be learned.

The use of metal organic frameworks (MOFs) as drug carriers is an emerging drug delivery strategy capable of delivering high drug cargoes. [15]MOFs offer targeted design toward specific disorders with stimuli-responsive delivery of their cargo upon activation by endogenous (i.e., pH, redox, and ATP) or exogenous stimuli (i.e., magnetic field, temperature, ions, pressure, light, and humidity).[16] In most cases, the MOF hosting the active pharmaceutical ingredient (API) is a carrier without biologic activity which will ultimately deconstruct inside the body, thus raising additional toxicity concerns.

In general, MOFs are gaining traction in the medical field, particularly for enhancing drug delivery via oral [17] and intravenous administration.[18] The use of MOFs as inhalation therapies for pulmonary disorders, though, remains largely unexplored, except for few very recent examples.[19, 20] However, these recent examples have utilized isoniazid (INH), a first-line anti-TB drug that is orally bioavailable and that is subject to the most prevalent drug resistance.[21] Thus, is is possible that administering INH as an aerosol treatment can not achieve the desired improvement in efficacy. These inhalable MOFs were also composed of additional excipient material that can lower overall activity. Finally, little aerodynamic characterization has been reported. This characterization can be helpful to fully evaluate the potential effectiveness of an inhaled therapy.[22]

The presently disclosed subject matter relates in one aspect to a spray drying procedure for preparing inhalable spray-dried MOF aerosols displaying a micrometric hollow spherical arrangement of bis(pyrazine-2-carboxylato)copper(II) nanocrystals (also referred to herein copper-pyrazinoate or Cu(POA)₂) with suitable aerodynamic features for pulmonary administration. The choice of Cu as the metal of the spray-dried MOF is based on its anti-TB potential.[23, 24] POA was selected because, as mentioned above, it is considered a prodrug of PZA, a current first line TB drug experiencing drug resistance from Mtb. [13, 25]

The presently disclosed subject matter also relates to spray-dried MOF compositions for the treatment and/or diagnosis of pulmonary diseases or disorders more generally. Thus, in some embodiments, the presently disclosed subject matter provides a spray-dried metal-organic framework (MOF) particle for treating and/or diagnosing a pulmonary disease or disorder, the spray-dried MOF comprising a metal ion (e.g., an ion of an alkaline metal, alkaline earth metal, transition metal or lanthanide) and an organic ligand coordinated to said metal ion, wherein said organic ligand comprises at least two metal coordination sites and wherein the organic ligand has a biological activity related to treatment and/or diagnosis of a pulmonary disease or disorder or where the organic ligand is a prodrug of a compound with such biological activity. In some embodiments, the presently disclosed subject matter provides a spray-dried metal-organic framework (MOF) particle for treating and/or diagnosing a pulmonary disease or disorder, the spray-dried MOF particle comprising: a metal ion, wherein the metal ion has a biological activity related to treatment of the pulmonary disease or disorder and/or a property useful for biomedical imaging; and an organic ligand coordinated to said metal ion, wherein said organic ligand comprises at least two metal coordination sites and wherein the organic ligand has a biological activity related to treatment of the pulmonary disease or disorder, is a prodrug of a therapeutic agent that has a biological activity related to treatment of the pulmonary disease or disorder, and/or has a property useful for biomedical imaging. Thus, in some embodiments, the presently disclosed spray-dried MOFs are entirely composed of components that have a therapeutic or diagnostic effect or utility. In some embodiments, the presently disclosed MOF comprises at least two APIs, a metal ion API and an organic molecule API.

In some embodiments, the metal ion can be any ion that has a therapeutic effect and/or a property useful in biomedical imaging (e.g., magnetic resonance imaging (MRI) or computed tomography (CT) imaging). In some embodiments, the metal ion has antimicrobial (e.g., antibacterial or antiviral) activity. In some embodiments, the metal ion can be paramagnetic or diamagnetic. In some embodiments, the metal ion can be an ion of an element having a high atomic number. In some embodiments, the metal ion is non-toxic. In some embodiments, the metal ion is an ion of a transition metal (i.e., scandium (Sc), yttrium (Y), titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), zinc (Zn), cadmium (Cd), and mercury (Hg)) or a lanthanide (i.e., lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu)). For example, in some embodiments, the metal ion is an ion of an element selected from Cu, Fe, Pd, Zn, Ag, Au, Mn, Co, Rh, Ni, Ta, Ti, W, Y, V, Pt, Gd, and Yb. In some embodiments, the metal ion is an ion of an element of the group comprising Cu, Fe, Pd, Zn, Ag, Au, Rh, Ni, Co, and Pt. In some embodiments, the metal ion is an ion of the group comprising Gd, Fe, and Mn.

Any suitable organic ligand having at least two metal coordination sites (i.e., an at least bidentate organic ligand) that has biological activity related to treating a pulmonary disease or disorder of interest, that is a prodrug of a molecule having such a biological activity, and/or that has a property useful for biomedical imaging can be used. Non-limiting functional groups that can act as metal coordination sites and that can be contained by the organic ligand according to the presently disclosed subject matter include, but are not limited to, —OH (including phenol —OH groups), —COOH, —CSSH, —NO₂, —B(OH)₂, —SO₃H, —Ge(OH)₃, —Sn(OH)₃, —Si(SH)₄, —Ge(SH)₄, —Sn(SH)₃, —PO₃H, —AsO₃H, —AsO₄H, —P(SH)₃, —As(SH)₃, —C₄H₂O—, —RSH, —RNH₂, —RNR—, —ROH, —RCN, —PO(OR)₂, and —RN₃, where R is hydrogen, alkyl, alkylene, preferably C1, C2, C3, C4 or C₅ alkylene, or aryl group, preferably comprising 1 or 2 aromatic nuclei. Accordingly, in some embodiments, the organic ligand includes at least two functional groups selected from hydroxyl (e.g., phenol), carboxylate, phosphonate, amino, azide, cyano, and heterocycle, e.g., a N-heterocycle, such as a pyridine, a pyrazine, or an azole. In some embodiments, the organic ligand comprises at least one carboxylate or carboxylic acid group. In some embodiments, the organic ligand comprises at least one N-heterocycle.

In some embodiments, the organic ligand is a selected from a compound known in the art as an anti-asthmatic, an antihistamine, an antitussive, a bronchodilator (e.g., an adrenergic bronchodilator (e.g., terbutaline, hexoprenaline, isoprenaline, salmeterol, etc.), an anticholinergic bronchodilator), a methylxanthine, a decongestant, an expectorant, a leukotriene modifier, a lung surfactant, a respiratory agent, an anti-infective (e.g., an antibacterial, antibungal or antiviral agent), a corticosteroid (e.g., beclomethasone, hydrocortisone, etc.), a mast cell stabilizer, a mucolytic, and/or a selective phosphodiesterase-4 inhibitor (e.g., theophylline) In some embodiments, the organic ligand is fluorescent or includes a radioisotope or otherwise has a property useful for MRI, CT, OI, PET or SPECT. In some embodiments, the organic ligand is an iodinated CT contrast agent or a derivative thereof. In some embodiments, the organic ligand is a known chemotherapeutic, e.g., used in the field to treat lung cancer. In some embodiments, the organic ligand is not azelaic acid. In some embodiments, the organic ligand is not isoniazid (INH). In some embodiments, the organic ligand is pyrazinoic acid.

In some embodiments, the spray-dried MOF particle has an average particle diameter between about 1 μm and about 5 μm (e.g., about 1.0, 1.5, 2.0, 2.5, 3.0, 3.5, 4.0, 4.5, or about 5.0 μm). In some embodiments, the average particle diameter is between about 2 μm and about 3 μm (e.g., about 2.0, 2.1, 2.2, 2.3, 2.4, 2.5, 2.6, 2.7, 2.8, 2.9, or about 3.0 μm).

In some embodiments, the spray-dried MOF particle is approximately spherical. In some embodiments, the particle is substantially hollow, i.e. has an opening in at least one portion of the particle, including for example in a center or middle portion.

In some embodiments, the spray-dried MOF particle comprises bis(pyrazine-2-carboxylato)copper(II) (Cu(POA)₂). In some embodiments, the Cu(POA)₂ is hydrated or solvated. In some embodiments, the MOF particle comprises Cu(POA)₂ ·2 H₂O. In some embodiments, the MOF particle comprises dehydrated Cu(POA)₂.

In some embodiments, the spray-dried MOF comprises at least two different metal ions (e.g., where both metal ions have a biological activity related to treatment of the pulmonary disease or disorder and/or have a property useful for biomedical imaging). Thus, in some embodiments, the spray-dried MOF comprises ions of at least two different elements selected from transition metals and lanthanides. In some embodiments, the spray-dried MOF comprises ions of at least two different elements selected from Cu, Fe, Pd, Zn, Ag, Au, Mn, Co, Rh, Ni, Ta, Ti, W, Y, V, Pt, Gd, and Yb. In some embodiments, the spray-dried MOF comprises at least two different types of ions where the ions are ions of elements selected from the group comprising Cu, Fe, Pd, and Zn (e.g., for the treatment of TB or another bacterial infection). In some embodiments the spray-dried MOF comprises both Ag and Pt ions (e.g., for the treatment of lung cancer). In some embodiments, the MOF comprises at least two types of ions selected from Ag, Cu, and Zn ions (e.g., for the treatment of a viral infection). In some embodiments, the spray-dried MOF comprises one metal ion having a therapeutic effect (e.g., a Pt, Au, Zn, Ag, Pt, Ti, V, Fe, Co, Ni, or Rh ion) and at least one metal ion having a property useful for MRI (e.g., a Gd, Fe, or Mn ion) or having a property useful for CT (e.g., Au, Gd, Y, Yb, W, Ta, or Pt). In some embodiments, the spray-dried MOF comprises both Cu and Gd. In some embodiments, the spray-dried MOF comprises Gd_{0.1}Cu_{0.9}(POA)₂.

In some embodiments, the MOF can include one or more metal ions that do not have a therapeutic and/or diagnostic effect (either in addition to one or more metal ions that have a therapeutic and/or diagnostic effect or as an alternative to the one or more metal ions that have a therapeutic and/or diagnostic effect. For example, some embodiments, the MOF can comprise one or more alkaline or alkaline earth metal ions (e.g., Li, Na, K, Rb, Cs, Mg, Ca, Sr, or Ba ions).

In some embodiments, the spray-dried MOF particle comprises at least two different organic ligands. In some embodiments, the at least two different organic ligands are each a polydentate organic ligand selected from the types of therapeutic and/or imaging agents listed above.

In some embodiments, the spray-dried MOF can comprise an additional therapeutic agent for treatment of the pulmonary disease or disorder embedded or sequestered in the MOF. In some embodiments, the spray-dried MOF can comprise one or more coating agents or layers covering or partially covering the outer surface of the MOF particle. The coating agent or layer can comprise a polymer to stabilize or protect the MOF or to provide biodegradable time-release of the APIs included in the MOF or targeting of the MOF to diseased cells or infectious agents. In some embodiments, the coating layer can comprise polyvinylpyrrolidone (PVP) or another water soluble or hydrophilic polymer. In some embodiments, the coating layer can comprise one or more polymers selected from the group including, but not limited to, poly(L-lactic acid) polymers, poly(D,L-lactide-co-glycolide polymers, polycaprolactone-polyethylene glycol diblock and triblock polymers, polylactic acid-polyethylene glycol diblock and triblock polymers, chitosan, chitin, hydroxybutyric acid polymers, polyanhydrides, polyesters, polyphosphazenes, polyphosphoesters, and styrene-maleic anhydride copolymers (e.g., LIPODISQ®, SigmaMillipore, Burlington, Massachusetts, United States of America).

In some embodiments, the presently disclosed subject matter provides a therapeutic and/or diagnostic composition comprising a spray-dried MOF of the presently disclosed subject matter. In some embodiments, the therapeutic and/or diagnostic composition is a therapeutic composition where the spray-dried MOF comprises at least two APIs, a metal ion API and an organic ligand API. In some embodiments, at least one of the metal ion or the organic ligand has a property useful for biomedical imaging.

In some embodiments, the metal ion of the spray-dried MOF of the therapeutic and/or diagnostic composition is an ion of a transition metal or a lanthanide. In some embodiments, the spray-dried MOF comprises at least one metal ion of an element selected from Cu, Fe, Pd, Zn, Ag, Au, Mn, Co, Rh, Ni, Ta Ti, W, Y, V, Pt, Gd, and Yb. In some embodiments, the spray-dried MOF comprises at least one organic ligand comprising at least two metal coordination sites and selected from an anti-asthmatic, an antihistamine, a antitussive, a bronchodilator, a decongestant, an expectorant, a leukotriene modifier, a lung surfactant, an anti-infective, a corticosteroid, a mast cell stabilizer, a mucolytic, a selective phosphodiesterase-4 inhibitor, or a combination thereof. In some embodiments, the spray-dried MOF comprises at least one organic ligand comprising at least two metal coordination sites and that has a property useful in biomedicial imaging.

In some embodiments, the therapeutic and/or diagnostic composition comprises an aerosolized form of the spray-dried MOF particle. In some embodiments, the aerosolized form of the spray-dried MOF particle has an aerodynamic particle size distribution (APSD) in a range suitable for pulmonary delivery or the therapeutic composition is otherwise suitable for delivery via inhalation. In some embodiments, the APSD is in a range between about 1 μm and about 5 μm. In some embodiments, the APSD is between about 2 μm and about 3 μm.

In some embodiments, the therapeutic and/or diagnostic composition is provided for use with an inhaler (e.g., a pressurized metered dose inhaler) or a nebulizer. In some embodiments, the therapeutic and/or diagnostic composition is provided for use with a dry powder inhaler (DPI).

In some embodiments, the therapeutic and/or diagnostic composition comprises one or more excipient or carrier. In some embodiments, the one or more excipient or carrier is selected from the group comprising a sugar, a peptide, a lipid and a surfactant. Suitable sugars for use in the presently disclosed therapeutic compositions include, but are not limited to, maltodextrin, mannitol, trehalose, lactose, and/or dextrose. Suitable peptides for use in the presently disclosed therapeutic composition include, but are not limited to, leucine, lysine, glycine, polyleucine (dileucine, trileucine), and/or polylysine (dilysine). The excipient and/or carriers can include lipids including but not limited to lecithin (phosphatidyl choline), phosphatidylethanolamine and/or magnesium stearate.

In some embodiments, the therapeutic and/or diagnostic composition is configured to treat or diagnose a pulmonary disease or disorder when administered by inhalation. In some embodiments, the pulmonary disease or disorder is selected from a bacterial infection (e.g., a Mycobacterium tuberculosis infection or an infection related to Legionnaires disease, whooping cough or bacterial pneumonia), a viral infection (e.g., a coronavirus infection, such as a COVID-19, MERS, or SARS infection; an infection related to Influenza A, B, or C; viral pneumonia; respiratory syncytial virus; swine flu; or avian flu), asthma, chronic obstructive pulmonary disorder (COPD), cystic fibrosis, emphysema, bronchitis, and lung cancer. However, the pulmonary disease or disorder can be any pulmonary disease or disorder that can be treated and/or diagnosed with the metal ion and/or organic ligand. For instance, the pulmonary disease that can be treated with the composition can be any pulmonary disease treatable using an anti-asthmatic, an antihistamine, a antitussive, a bronchodilator, a decongestant, an expectorant, a leukotriene modifier, a lung surfactant, an anti-infective, a corticosteroid, a mast cell stabilizer, a mucolytic, a selective phosphodiesterase-4 inhibitor. In some embodiments, the therapeutic and/or diagnostic composition is configured to treat tuberculosis (TB). In some embodiments, the therapeutic and/or diagnostic composition comprises an aerosolized formulation of an MOF particle comprising bis(pyrazine-2-carboxylato)copper(II) (Cu(POA)₂). In some embodiments, the therapeutic and/or diagnostic composition comprises an aerosolized formulation of an MOF particle comprising Gd_{0.1}Cu_{0.9}(POA)₂.

### III. Methods of Treatment

In some embodiments, the presently disclosed subject matter provides a method of treating a pulmonary disease or condition in a subject in need thereof, wherein the method comprises administering to the subject a therapeutically effective amount of a MOF comprising (a) at least one metal ion and (b) at least one organic ligand, wherein said at least one organic ligand comprises at least two metal coordination sites (optionally wherein the at least one metal ion and the at least one organic ligand each have a biological activity related to treating the pulmonary disease or disorder and/or a property useful for biomedical imaging), subject to the proviso that at least one of the at least one metal ion and the at least one organic ligand has a biological activity related to treating the pulmonary disease or disorder. In some embodiments, the at least one metal ion and the at least one organic ligand both have a biological activity related to treating the pulmonary disease or disorder. In some embodiments, the MOF is prepared via spray drying. In some embodiments, the MOF is delivered or administered as an aerosol.

In some embodiments, the presently disclosed subject matter provides a method of treating a pulmonary disease or disorder in a subject in need of treatment thereof, the method comprising: providing a subject in need of treatment (e.g., a subject diagnosed with the pulmonary disease or disorder); providing an inhalable formulation of a spray-dried MOF particle comprising (a) a metal ion (optionally wherein the metal ion has a biological activity related to treatment of the pulmonary disease or disorder); and (b) an organic ligand coordinated to said metal ion, wherein said organic ligand comprises at least two metal coordination sites and wherein the organic ligand has a biological activity related to treatment of the pulmonary disease or disorder or is a prodrug of a therapeutic agent that has a biological activity related to treatment of the pulmonary disease or disorder; and administering an effective amount of the inhalable formulation of the spray-dried MOF particle to the subject by inhalation, wherein the pulmonary disease or disorder in the subject is treated. In some embodiments, the inhalable formulation of the spray-dried MOF particle comprises an aerosolized form of the MOF particle. The aerosolized form of the spray-dried MOF particle can have an aerodynamic particle size distribution (APSD) in a range suitable for pulmonary delivery. For example, in some embodiments, the APSD is about 1 μm to about 5 μm. In some embodiments, the APSD is about 2 μm to about 3 μm. In some embodiments, the spray-dried MOF microparticle is hollow.

In some embodiments, the subject is a human or other mammalian subject. In some embodiments, the subject has a disease or disorder selected from the group including, but not limited to, a bacterial infection (e.g., a Mycobacterium tuberculosis infection or an infection related to Legionnaires disease, whooping cough or bacterial pneumonia), a viral infection (e.g., a coronavirus infection, such as a COVID-19, MERS, or SARS infection; an infection related to Influenza A, B, or C; viral pneumonia; respiratory syncytial virus; swine flu; or avian flu), asthma, chronic obstructive pulmonary disorder (COPD), cystic fibrosis, emphysema, bronchitis, lung cancer, or any other pulmonary disease or disorder treatable by the organic ligand and/or metal ion. In some embodiments, the subject has TB. In some embodiments, the TB in the subject comprises multiple drug resistant (MDR) TB or extensively drug resistant (XDR) TB.

In some embodiments, the spray-dried MOF comprises a metal ion of an element selected from Cu, Fe, Pd, Zn, Ag, Au, Mn, Co, Rh, Ni, Ti, V, and Pt. In some embodiments, the spray-dried MOF comprises an organic ligand having at least two metal coordination sites and which selected from the group comprising an anti-asthmatic, an antihistamine, an antitussive, a bronchodilator, a decongestant, an expectorant, a leukotriene modifier, a lung surfactant, an anti-infective, a corticosteroid, a mast cell stabilizer, a mucolytic, a selective phosphodiesterase-4 inhibitor, or a combination thereof.

In some embodiments, the spray-dried MOF particle comprises an organic ligand comprising at least one carboxylate or carboxylic acid group. In some embodiments, the spray-dried MOF particle comprises an organic ligand comprising at least one N-heterocycle. In some embodiments, the spray-dried MOF comprises at least two types of therapeutic metal ion. In some embodiments, the spray-dried MOF comprises at least two types of therapeutic organic ligand.

In some embodiments, the organic ligand is POA. In some embodiments, the spray-dried MOF particle comprises or consists of bis(pyrazine-2-carboxylato)copper(II) (Cu(POA)₂). In some embodiments, the spray-dried MOF particle further comprises a second metal ion (i.e., a metal ion in addition to a Cu ion). In some embodiments, the spray-dried MOF particle further comprises a metal ion selected from Fe, Pd, and Zn. In some embodiments, the second metal ion can be a metal ion having a property useful in biomedical imaging, e.g., as a contrast MRI agent. In some embodiments, the second metal ion can be Gd.

With respect to the methods of the presently disclosed subject matter, a preferred subject is a vertebrate subject. A preferred vertebrate is warm-blooded; a preferred warm-blooded vertebrate is a mammal. The subject treated by the presently disclosed methods is desirably a human, although it is to be understood that the principles of the presently disclosed subject matter indicate effectiveness with respect to all vertebrate species which are to be included in the term “subject.” In this context, a vertebrate is understood to be any vertebrate species in which treatment of a pulmonary disease or disorder (e.g., TB or another bacterial or viral infection) is desirable. As used herein, the term “subject” includes both human and animal subjects. Thus, veterinary therapeutic uses are provided in accordance with the presently disclosed subject matter.

As such, the presently disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economical importance to humans. Thus, also provided is the treatment of livestock, including, but not limited to, domesticated swine, ruminants, ungulates, horses (including racehorses), poultry, and the like. In some embodiments, the subject is a human.

The therapeutically effective amount of a composition can depend on a number of factors. For example, the species, age, and weight of the subject, the precise condition requiring treatment and its severity, the nature of the formulation, and the route of administration are all factors that can be considered.

A spray-dried MOF of presently disclosed subject matter can also be useful as adjunctive, add-on or supplementary therapy for the treatment of the above-mentioned diseases/disorders. Said adjunctive, add-on or supplementary therapy means the concomitant or sequential administration of a compound of the presently disclosed subject matter to a subject who has already received administration of, who is receiving administration of, or who will receive administration of one or more additional or “second” therapeutic agents for the treatment of the indicated conditions, for example, one or more known anti-bacterial, anti-viral, or anti-cancer agents. In some embodiments, the spray-dried MOF is an adjunctive therapy for a second therapeutic agent being administered orally, subcutaneously, intravenously or via any other suitable method.

### VI. Methods of Diagnosing a Pulmonary Disease or Disorder

In some embodiments, the presently disclosed subject matter provides a method of diagnosing a pulmonary disease or disorder using a spray-dried MOF. In some embodiments, the spray-dried MOF comprises at least one metal ion of an element having a property useful in biomedical imaging (e.g., CT or MRI) and/or at least one at least bidentate organic ligand having a property useful in biomedical imaging.

In some embodiments, the presently disclosed subject matter provides a method of diagnosing a pulmonary disease or disorder using a spray-dried MOF, the method comprising: providing a subject suspected or at risk of having a pulmonary disease or disorder; providing an inhalable formulation of a spray-dried MOF particle of the presently disclosed subject matter that includes at least one component (i.e., a metal ion or an organic ligand) that has a property useful for biomedical imaging); administering an effective amount of the inhalable formulation of the spray-dried MOF particle to the subject by inhalation; imaging at least a portion of at least one lung of the subject; and analyzing results of the imaging and finding evidence of the presence of a pulmonary disease or disorder; thereby diagnosing the pulmonary disease or disorder in the subject.

In some embodiments, the spray-dried MOF particle comprises a metal ion of an element selected from Gd, Fe, Mn, Au, Y, Yb, Ni, Cu, W, Ta, Pt, and Ti. In some embodiments, the metal ion is a Gd ion, a Fe ion, or a Mn ion.

In some embodiments, the organic ligand is an iodinated contrast agent, a radiologic contrast agent or comprises a fluorescent moiety.

In some embodiments, the imaging comprises performing MRI or CT.

### V. Methods of Preparing MOFs and Aerosols Thereof

The presently disclosed MOFs can be prepared using synthetic methodology known in the art. For example, the MOFs can be prepared by the methods described hereinbelow or variations thereof that will be apparent to persons skilled in the art based on the present disclosure.

Generally, MOFs can be prepared by hydrothermal or solvothermal techniques, where crystals are slowly grown from a solution of a metal precursor, such as a metal salt, and bridging ligands. U.S. Pat. No. 9,352,489, the disclosure of which is incorporated herein by reference in its entirety describes a method of preparing a MOF using spray-drying, the method generally comprising spray drying at least one metal ion and at least one organic ligand (which is at least bidentate) in the presence of a solvent and then collecting the formed MOF crystals. As used herein the term “spraying” (also known as atomizing) refers to the process of forming a spray, which is a dynamic collection of drops dispersed in a gas. The term “spray dryer” refers to a device known in the art where a liquid input stream is sprayed in small droplets through a nozzle (or atomizer) into a hot gas stream.

The metal ion for use in preparing the presently disclosed spray-dried MOFs can be selected from any metal ion that has or is suspected of having a biological activity related to treating a pulmonary disease or disorder of interest or that has a property related to a biomedical imaging technique. For example, MRI can involve the use of paramagnetic metal ions, e.g., Ti, Mn, Fe, Co, Ni, Cu, and Gd, while CT can involve the use of metal ions such as Au, Gd, Y, Yb, W, Ta, and Pt. In some embodiments, the metal ion can be a metal ion that has shown in vitro or in vivo activity related to disease treatment. In some embodiments, the metal ion is an ion of an element selected from the group including, but not limited to, Cu, Fe, Pd, Zn, Ag, Au, Mn, Co, Rh, Ni, Ti, V, and Pt, or to a combination of such ions. In some embodiments, MOF formation can take place by reacting more than one metal ion (e.g., by reacting two, three, four, five, six or more metal ions). Usually, the metal ion is provided in the form of a salt. Non-limiting examples of metal salts for use in the presently disclosed subject matter are nitrates, chlorides, sulphates, acetates, acetylacetonates, bromides, carbonates, tartrates and perchlorates. In some embodiments, the one or more metal ions are reacted with an organic ligand that is at least bidentate (e.g., bidentate, tridentate, tetradentate, etc.) and that has biological activity related to treating a pulmonary disease or disorder, that is a prodrug of a compound that has biological activity related to treating a pulmonary disease or disorder, such as a compound in one of the drug classes described hereinabove, and/or that has a property useful for biomedical imaging. Thus, the organic ligand, which can also be referred to as an organic bridging ligand, includes at least two functional groups that can coordinate to metal ions. These functions groups can be the same or different. The organic ligand thus can coordinatively bond to at least two metal ions, which can be the same type of metal ion or can be different types of metal ions. In some embodiments, the MOF can be formed by reacting two or more organic ligands that are each at least bidentate with one or more metal salts. In some embodiments, the MOF can be formed by reacting two more metal salts with one or more organic ligands that are each at least bidentate.

The ability to coordinate metal ions can be conferred by certain functional groups. Non-limiting functional groups that can be contained by the organic ligand to form a MOF according to the presently disclosed subject matter include, for example, —OH, —COOH, —CSSH, —NO₂, —B(OH)₂, —SO₃H, —Ge(OH)₃, —Sn(OH)₃, —Si(SH)₄, —Ge(SH)₄, —Sn(SH)₃, —PO₃H, —AsO₃H, —AsO₄H, —P(SH)₃, —As(SH)₃, —C₄H₂O—, —RSH, —RNH₂, —RNR—, —ROH, —RCN, —PO(OR)₂, and —RN₃, where R is hydrogen, alkyl, alkylene, preferably C1, C2, C3, C4 or C5 alkylene, or aryl group, preferably comprising 1 or 2 aromatic nuclei. Accordingly, in some embodiments, the organic ligand includes at least two functional groups selected from hydroxyl, carboxylate, phosphonate, amino, azide, cyano, and heterocycle, e.g., a N-heterocycle, such as a pyridine, a pyrazine, or an azole. In some embodiments, the organic ligand comprises at least one carboxylate group.

In some embodiments, a solvent is present for the reagents to form the MOF. The term “solvent” relates to individual solvents and also to mixtures of different solvents. The solvent can be any aqueous or non-aqueous solvent. In some embodiments, mixtures of two or more solvents are used. In some embodiments, the solvent is selected from the group consisting of water, (C1-C6)-alcohols, (C5-C7)-alkanes, alkenes, (C3-C8)-cycloalkanes, N,N-dimethyl formamide (DMF), N,N-diethyl formamide (DEF), dimethyl sulfoxide (DMSO), dioxane, chloroform, dichloromethane, diethyl ether, acetonitrile, toluene, benzene, tetrahydrofuran (THF), chlorobenzene, ethylene glycol, and mixtures thereof. In some embodiments, the alcohol is selected from methanol, ethanol, and isopropanol. In some embodiments, the alkane is selected from hexane, heptane, and pentane. In some embodiments, the solvent is or comprises a mixture of water and an alcohol (e.g., ethanol). In some embodiments, the solvent comprises a mixture of 80% (by volume) water and 20% (by volume) ethanol.

In some embodiments, a solution comprising both the at least one metal ion and the at least one organic ligand which is at least bidentate can be sprayed into the spray dryer apparatus in the presence of a solvent. The moment this solution is sprayed into the spray dryer the reaction and the drying take place and dry MOF crystals are formed. Thus, in some embodiments, the process of preparing the MOF comprises spraying a liquid solution containing both the at least one metal ion and the at least one organic ligand which is at least bidentate into a spray dryer in the presence of a solvent. The solvent is usually included in the sprayed solution comprising the at least one metal ion and the at least one organic ligand. Additionally, more solvent can be simultaneously sprayed through a different nozzle.

Alternatively, at least two different solutions, one containing the at least one metal ion and the other one containing the at least one organic ligand, can be simultaneously fed to the spray dryer through separate nozzles or a three-fluid nozzle. Thus, in some embodiments, the process of preparing the MOF comprises simultaneously spraying two liquid solutions, one containing the at least one metal ion and another containing the at least one organic ligand, into a spray dryer in the presence of a solvent. The solvent can be fed to the reaction contained in either the metal ion or the organic ligand solution, or it can be contained in both solutions. Additionally, more solvent can be simultaneously sprayed through a different nozzle or a multi-fluid nozzle.

In some embodiments, the process of forming the spray-dried MOF can further comprise the addition of a base. The base can be sprayed into the spray dryer together with one of the reactants, together with the solvent, it can be simultaneously sprayed through a different nozzle, or a combination thereof. In some embodiments, the base is not contained in the liquid solution containing both the metal ion and the at least bidentate organic ligand. In principle any base can be used. However, in some embodiments, the base is selected from the group consisting of metal alkaline or earth alkaline hydroxides, amines, metal alkaline or earth alkaline carbonates, metal alkaline or earth alkaline acetates, pyridines, azoles, diazines, and mixtures thereof.

Any regular spray dryer can be used to produce dry crystalline MOFs according to the presently disclosed subject matter. In some embodiments, a large industrial spray dryer is used. Such industrial spray dryers can produce very large quantities of MOFs according to the present method in short times.

In some embodiments, air can be used as a hot gas stream during spray drying, however, other gases can also be employed, such as nitrogen. Industrial spray dryers can have large capacity drums for product collection. In some embodiments, the spray dryer drum is coupled to a powder-removing device. The device can remove the formed MOF powder at regular intervals, making place for newly synthesized MOF. Thus, in some embodiments, the process for the preparation of a dry crystalline MOF according to the presently disclosed subject matter is a continuous process.

The preparation of the MOFs of the presently disclosed subject matter can proceed at mild conditions, such that it is easily amenable to large-scale industrial production. The reaction temperature inside the spray dryer can be between about 20° C. and about 350° C. In some embodiments, the temperature can be between about 50° C. and about 250° C. In some embodiments, the temperature can be between about 50° C. and about 200° C. (e.g., about 50, 60, 70, 80, 90, 100, 110, 120, 130, 140, 150, 160, 170, 180, 190, or about 200° C.).

In some embodiments, the inlet temperature is between about 100° C. and about 250° C. In some embodiments, the inlet temperature is between about 120° C. and about 180° C. In some embodiments, the inlet temperature is about 150° C. or about 180° C.

The person skilled in the art is aware that the reaction parameters can be optimized for each MOF product and each spray-dryer. For instance, reagent concentrations, choice of solvent, choice and/or inclusion of base, gas spray flow, drying gas flow, feed flow and temperature, among others, are parameters that can be adjusted or optimized without departing from the scope of the instant disclosure.

In some embodiments, the feed gas used in preparing the presently disclosed mofs is nitrogen and the feed rate for the gas is between about 200 and about 1200 liter per hour (L/hr). In some embodiments, the concentration of the organic ligand is between about 0.5 and about 5 milligrams per mL (in a solution comprising water and/or alcohol). In some embodiments, the concentration of the organic ligand is between about 1 and about 3 mg/mL. In some embodiments, a molar excess of the organic ligand is used compared to the metal ion. In some embodiments, the ratio of metal ion to organic ligand is at least 1:1.2. In some embodiments, the ratio of metal ion to organic ligand is about 1:2.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Materials and Methods

Materials: All chemicals were used as received from Sigma-Aldrich (St. Louis, Missouri, United States of America) without further purification. Cu(NO₃)₂·2.5H₂O and pyrazinoic acid (HPOA).

Synthesis of bulk Cu(POA): 0.5 g Cu(NO₃)₂·2.5 H₂O and 0.533 g HPOA were dissolved in 360 μmL of water. Light blue crystals attributed to Cu(POA)₂·2H₂O or hydrated phase are formed after approximately 1 hour at room temperature (RT) and in few minutes at 60° C.

Spray drying Cu(POA)₂: A BÜCHI B-290 spray dryer (BÜCHI Labortechnik AG, Flawil, Switzerland) equipped with standard cyclone and two-fluid nozzle (inner orifice=0.7 μmm, outer orifice=1.5 μmm) was used to manufacture microparticulate dry powders. A dehumidifier was placed at the inlet of the aspirator. Aspiration rate (75%; ˜30 μm³/h) and solution pump speed (15%; ˜5 μmL/min) were kept constant throughout all experiments. The reagents were added to 80%:20% DI H₂O:EtOH (by volume) in a 1:2 Cu:POA molar ratio (0.5 g Cu(NO₃)₂·2.5 H₂O and 0.533 g POA). Immediately prior to spray drying, the POA solution was poured gently into the Cu solution. Concentration of this feed solution was labelled as high, medium, or low based on the total solution volume: 180 μmL (3 mg/mL POA), 360 μmL (1.5 mg/mL POA), or 540 μmL (1 mg/mL POA) where the POA and Cu solutions were each 90 μmL, 180 μmL, and 270 μmL respectively. The inlet temperature was either set to 180° C. (high) or 150° C. (low) resulting in outlet temperature of 88-90° C. and 72° C. (low N2 flow) or 65° C. (high N2 flow) respectively. Atomization gas (N2) flow was either 283 L/hour (20 μmm on B-290 rotameter, 15 kPA pressure drop) or 1052 L/hour (50 μmm on B290 rotameter, 75 kPa pressure drop).

Spray-drying of Gd_{0.1}Cu_{0.9}(POA)₂: Spray-dried Gd_{0.1}Cu_{0.9}(POA)₂ MOFs were prepared in a manner analogous to the preparation of the spray-dried Cu(POA)₂, except replacing the Cu solution with a solution comprising a 1:9 molar ratio of Gd(NO₃)₃ and Cu(NO₃)₂.

Characterization: X-ray powder diffraction (XRPD; Bruker AXS GmbH., Karlsruhe, Germany) was used to confirm the crystalline structure of MOF materials. XRPD patterns were recorded using a Panalytical Empyrean X-ray diffractometer with Cu Kα radiation (λ=1.54778 Å). Attenuated Total Reflection (ATR) infrared spectroscopy measurements were performed in the range of 4000-400 cm⁻¹ with a Perkin Elmer Spectrum 100 FTIR spectrometer (Perkin Elmer, Waltham, Massachusetts, United States of America). Scanning electron microscopy (SEM) images were acquired on a FEI Quanta 200 FEG Analytical Scanning Electron Microscope (Hillsborough, Oregon, United States of America) using a beam energy of 15 k. Laser diffraction was performed using a Malvern Mastersizer 2000 (Malvern Panalytical, Malvern, United Kingdom) with the Sirocco 2000 dry powder system. Approximately 100-150 mg of powder was loaded on to the sample tray and fed into the instrument at a feed ratio of 40% and dispersive air pressure of 2 bar. Triplicate measurements were taken for 2.5 seconds after the obscuration limit was reached (0.5-10) and in between 5 second background checks. Thermogravimetric analysis (TGA) was measured on a Q50, TA Instruments (New Castle, Delaware, United States of America) under air at 3° C./min. Transmission electron microscopy (TEM) images were acquired on a Hitachi H-7000 100 keV transmission electron microscope (Hitachi, Ltd., Tokyo, Japan) equipped with AMT digital camera and Kevex energy dispersive x-ray detector (EDX) with 4pi software.

Inertial impaction was performed using a Next Generation Impactor (NGI; TSI Corp., Shoreview, Minnesota, United States of America). Stages of the impactor were precoated with 1% silicone oil in hexanes (w/w) and the pre-separator was filled with 15 mL DI H₂O. A nominal mass of 10 mg Cu(POA)₂ powder was loaded into a #3 hydroxypropylmehtylcellulose (HPMC) capsule. The capsule was placed in a RS01 inhaler (Plastiape S.p.A., Osnago, Italy), pierced, and the inhaler was inserted into the NGI inlet mouthpiece adapter. The solenoid-controlled vacuum in line with the NGI was set to 60 L/min for 4 seconds and then turned on to begin the experiment. NGI characterization was performed in triplicate. All stages of the NGI, the inlet, inhaler, and capsule were washed with 10 μmL DI H₂O and assayed for POA content at 269 nm vis UV spectroscopy (SynergyMX, BioTek, Winooski, Vermont, United States of America). Cu content was quantified via inductively coupled plasma atomic emission spectroscopy (ICP-OES). An aliquot of the NGI collections were diluted 1:1 with 1% HNO₃ and vortex mixed. The samples were then loaded into an iCAP 7600 ICP-OES (Thermo Scientific, Waltham, Massachusetts, United States of America) instrument autosampler. The ICP-OES measures copper by detecting the characteristic wavelengths emitted by the copper in the argon plasma and comparing the response to a standard curve. Aerodynamic particle size distributions (APSD) were generated for both Cu and POA by plotting mass collected vs stage cutoff diameter. Mass median aerodynamic diameter (MMAD) was calculated by plotting the cumulative percentage of Cu or POA mass deposited in the NGI stages (y-axis), using a probability scale, against the corresponding cutoff diameter (x-axis) and applying a log-linear fit on either side of 50% cumulative mass. Geometric standard deviation (GSD) was calculated by the square root of the ratio of the particle size one standard deviation above and below the median particle size (84^{th} and 16^{th} percentile, respectively, or 1 and −1 on a probit scale). Fine particle fraction of the emitted (FPFED) dose was calculated as a ratio of the sum of drug mass collected below 4.46 μm (stage 3 to the micro-orifice filter) to the mass collected at the inlet of the NGI and below.

Dosator: All dosator supplies were purchased from McMaster Carr (Elmhurst, Illinois, United States of America). Manufacture and filling procedure was based on previous literature methods.[5] Briefly, the luer portion of a 21 G 2.54 cm blunt stainless steel needle was sanded down until ˜2-3 μmm of material remained (note a smaller needle was inserted into the 21 G to remove excess after sanding). This needle was tamped 1-5 times into a Cu(POA)₂ dry powder bed (˜35 mg) held in the bottom of a 0.5 conical tube. Lastly, this powder filled steel needle was inserted horizontally into a 20 G 5.08 cm Teflon needle. The powder was delivered into a small vial (equipped with a large needle protruding through a rubber bung for dosator access) with a volume of HNO₃ (pH ˜3.5) via 1 μmL syringe (pre filled with 0.3 μmL of air before attaching to the needle-in-needle setup). Cu and POA contents were quantified as above.

### Example 2

### Cu(POA)₂ MOFs

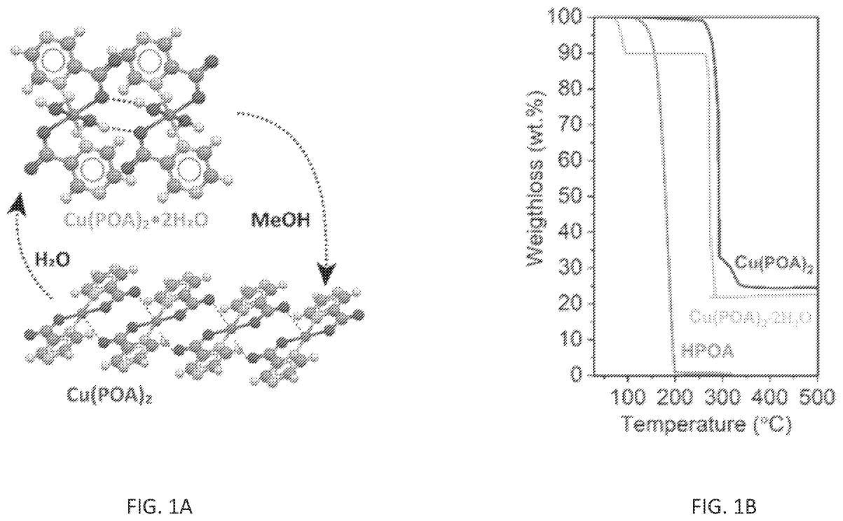

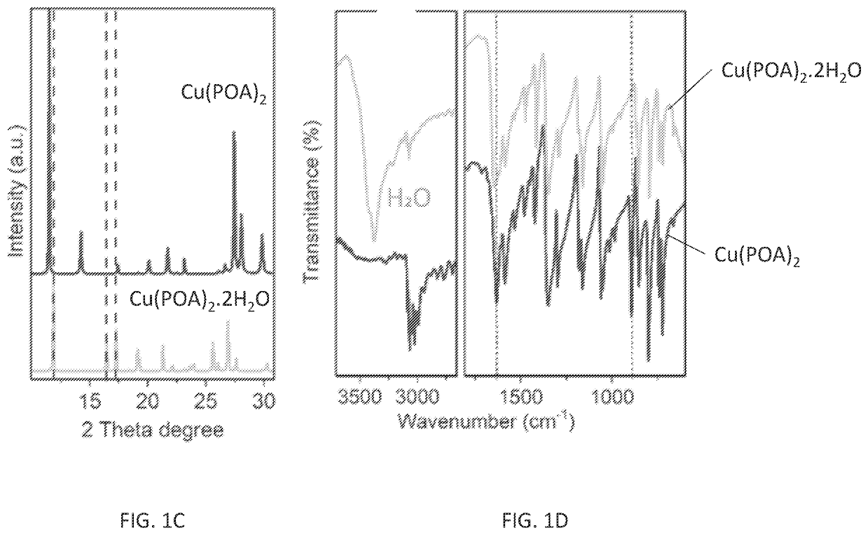

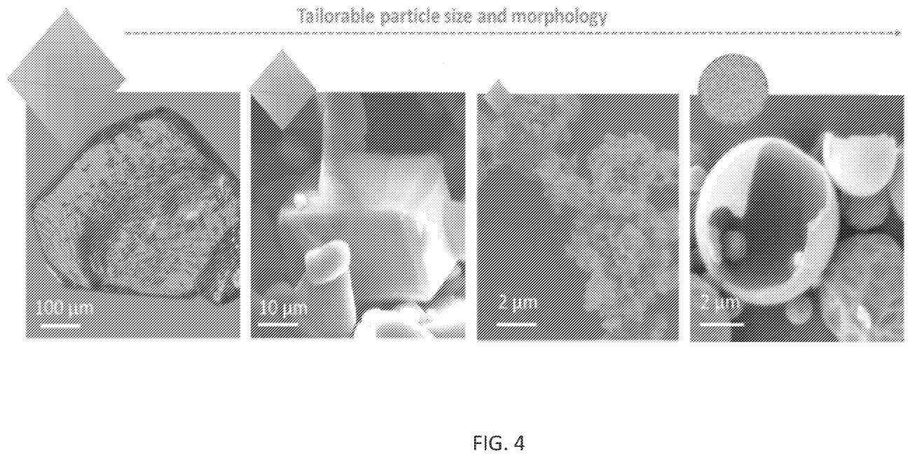

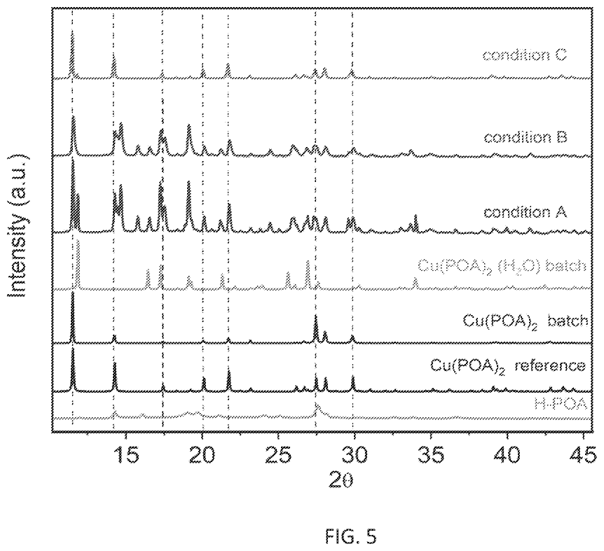

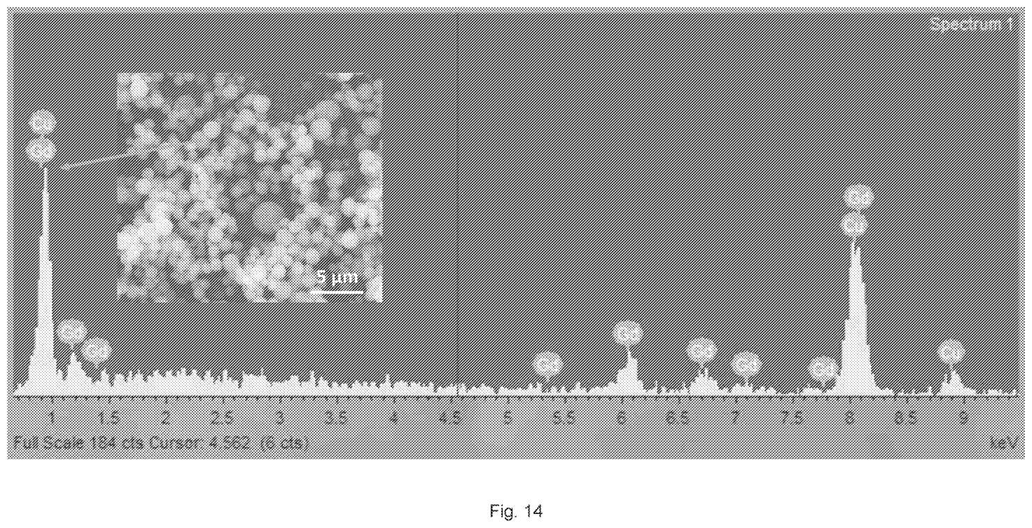

As shown in FIG. 1A, the combination of Cu salts (i.e. nitrate and acetate) with pyrazinoic acid (HPOA) in aqueous solution leads to light blue Cu(POA)₂·2H₂O bulk crystallites at room temperature after few hours (see FIG. 4). Thermogravimetric (TGA) analysis (see FIG. 1B) reveals the higher thermal stability of the Cu(POA)₂ MOF (up to 300° C.) compared to free POA (100-200° C.). The loss of ca 10 wt. % below 100° C. is attributable to crystallized water leading to a dark blue anhydrous Cu(POA)₂ crystalline phase, which can also be instantaneously obtained by exposing the hydrated phase to various organic solvents (i.e. MeOH, EtOH and DMF). X-ray powder diffraction (XRPD) analysis reveals two different crystalline structures for hydrated and dehydrated phases. See FIG. 1C. According to the literature [26], dehydrated crystalline phase displays a one-dimensional linear structure built from Cu (II) ions linked together by four oxygen atoms and two nitrogen atoms of POA molecules in a hexahedral coordination geometry. Conversely, the initial hydrated phase (Cu(POA)₂·2(H₂O)) shows six coordinated (distorted octahedral) Cu (II) ions with ligand and axially coordinated water molecules crystallizing in a hydrogen-bonded lattice. See FIG. 1A. Fourier-transform infrared spectroscopy (FTIR) analysis also clearly distinguishes between the two phases, as an absorption band attributed to coordinated water at 3.400 cm⁻¹ can be seen for the hydrated form in FIG. 1D. N2 isotherms for both phases revealed the non-porous nature of the materials. Soaking the hydrated and the anhydrous Cu(POA)₂ crystalline phases in MeOH and water, respectively, reveals a fully reversible dissolution-crystallization process, as represented by the scheme in FIG. 1A. FIG. 14 shows an SEM image and the EDS analysis of a MOF particle comprising two different metal ions, Gd and Cu.

Spray drying techniques have been widely used to prepare dry powders for therapeutic inhalation applications.[27,28] Generally, microparticles having a mass median aerodynamic diameter (MMAD) between 1-5 μm are considered respirable.[29] In particular, MMAD 2-3 μm can be considered particularly useful for pulmonary delivery and alveolar macrophage uptake.[20,30,31] Accordingly, with an eye toward in enhancing anti-TB drugs with Cu, Cu-pyrazinoic acid MOFs were spray dried and their resulting aerodynamic performance characterized. Spray drying has been demonstrated as a versatile continuous flow methodology to assemble nanoMOFs into micrometric hollow spherical superstructures.[32]

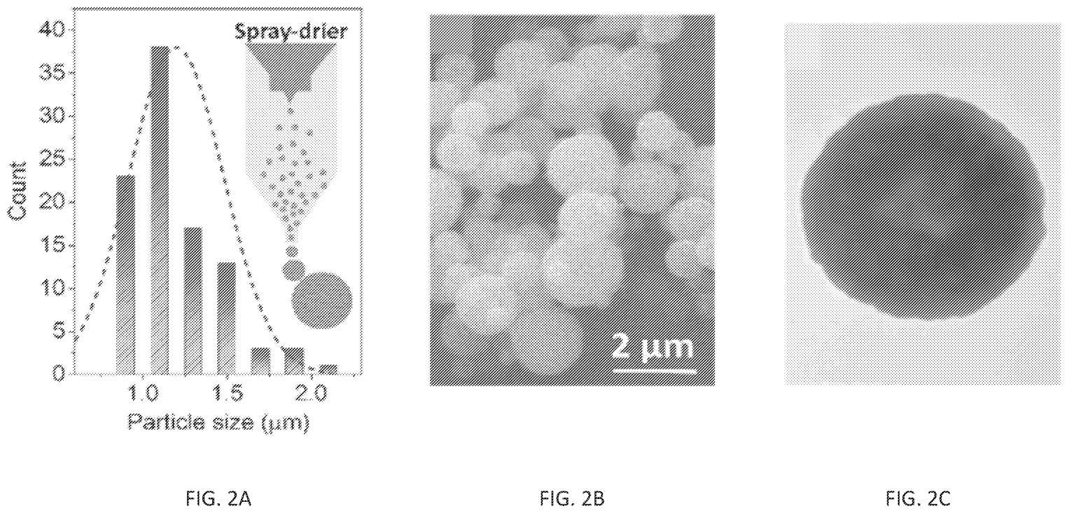

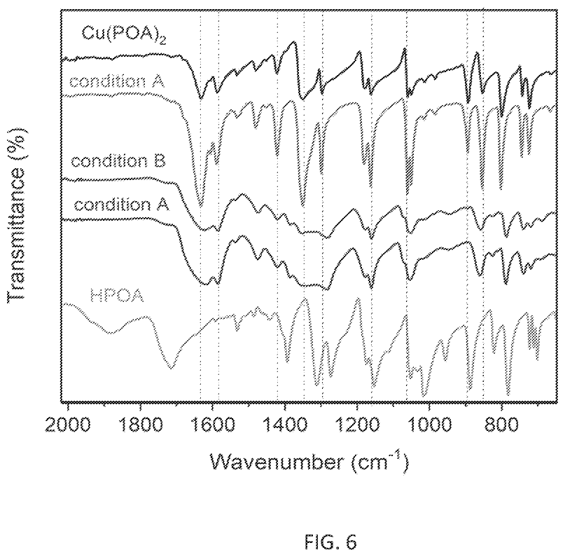

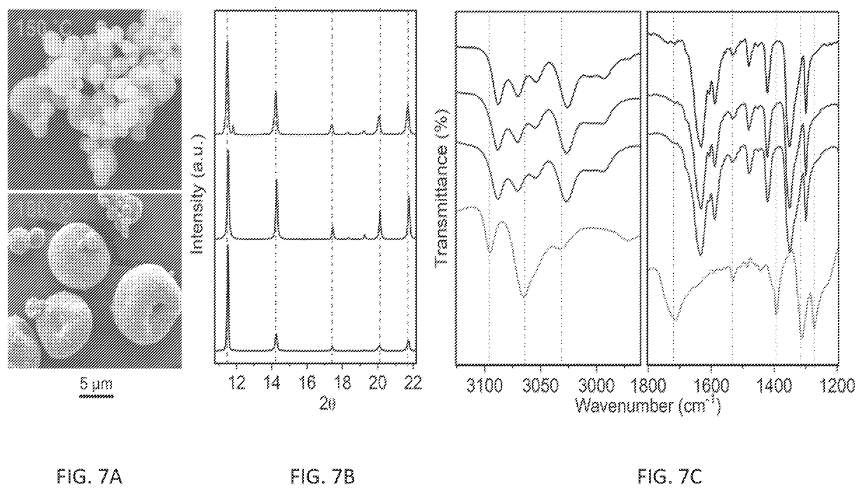

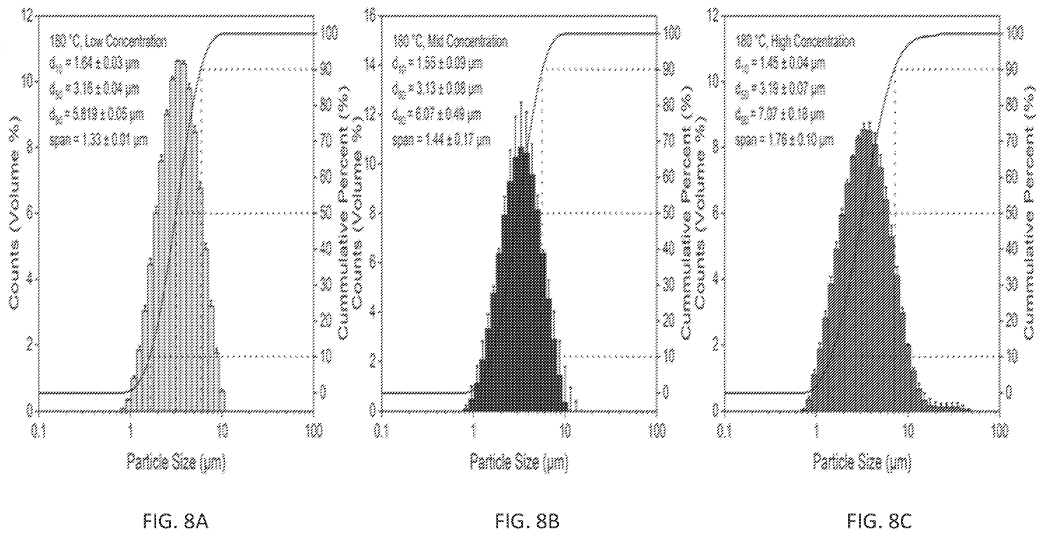

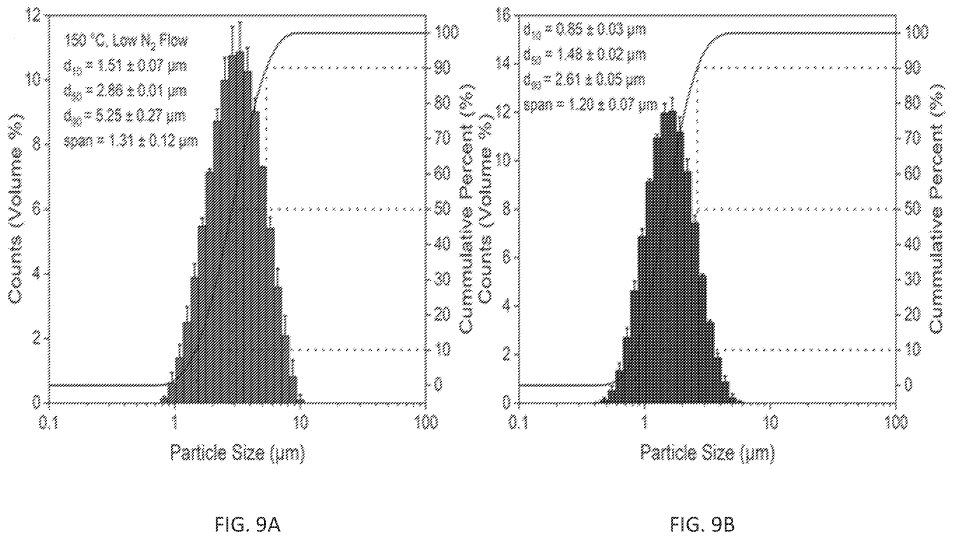

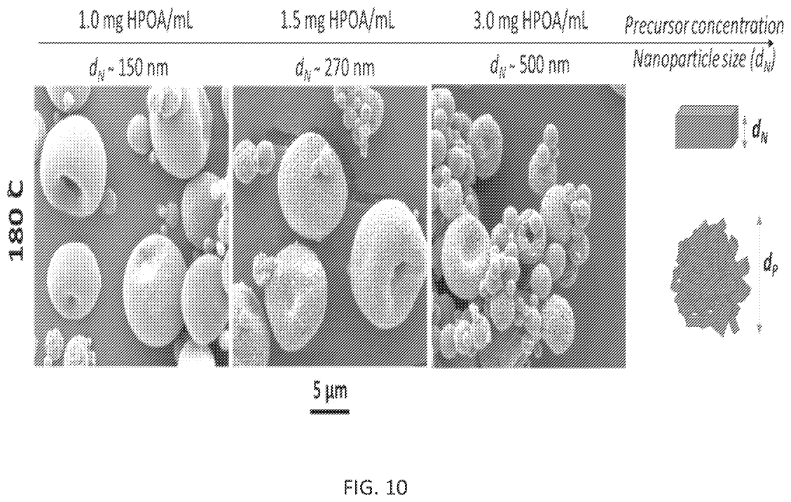

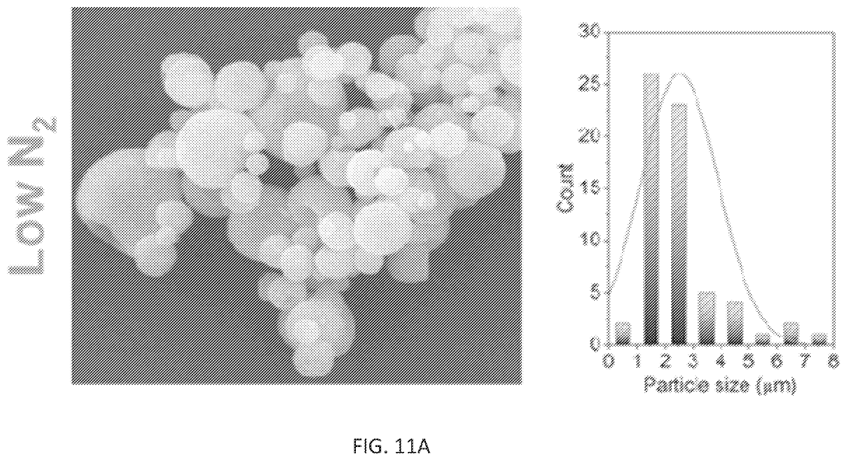

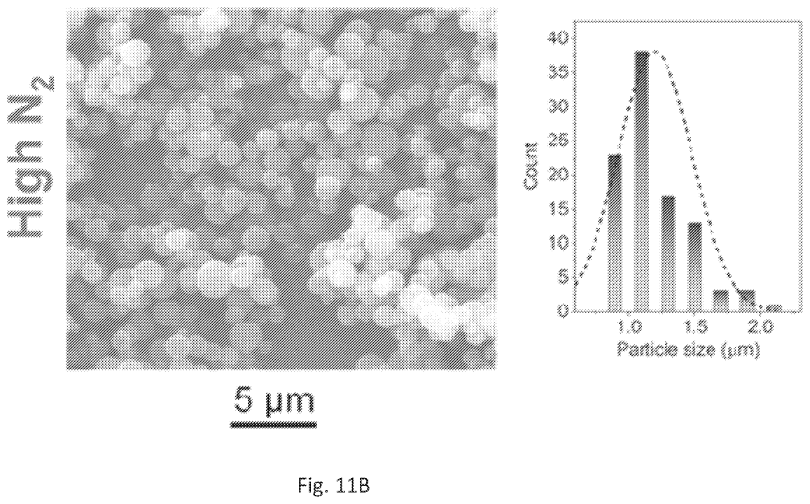

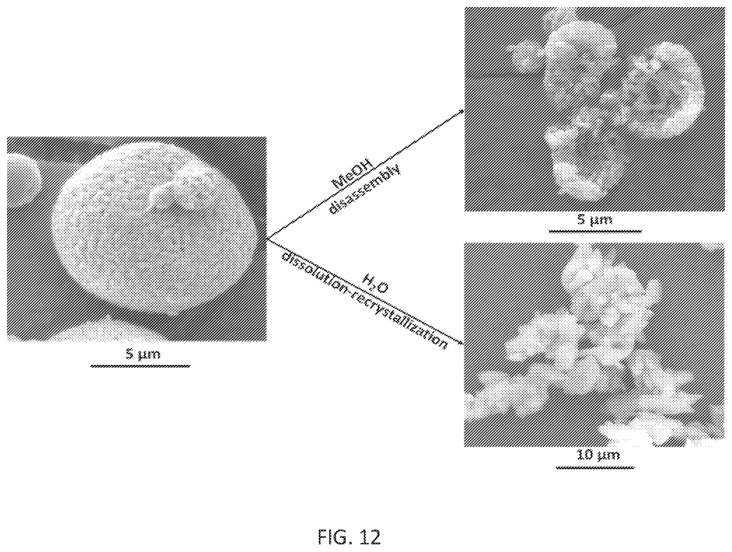

A systematic study of the effect of various spray drying parameters on the final properties of Cu pyrazinoic acid MOF aerosols was carried out to meet the targeted aerodynamic requisites (MMAD=2-3 μm). The resulting morphology, crystallinity, particle size, aerodynamic features, and composition of the MOF aerosols were examined after adjusting Cu:POA ratio, solvent mixture (EtOH:H₂O), precursor concentration, temperature, and atomizing gas flow rate. The use of 1:2 Cu:POA ratio is avoids the concomitant formation of non-reacted copper species (i.e. Cu(OH)₂) or free HPOA aggregates, attributed to the additional diffraction peaks (i.e. between 15 and 25°) and IR band associated to HPOA (i.e. free carboxylic acid at 1.700 cm⁻¹), respectively. See FIGS. 5 and 6. Addition of EtOH (20%) to the aqueous precursor solution promotes copper-pyrazinoate dehydration during the spray dryer synthesis, as pure water leads to a mixture of both crystalline phases. See FIG. 5. Higher Buchi B-290 inlet temperatures (180° C. vs 150° C.) promote the formation of larger aerosol MOF spheres. See FIG. 7. Laser diffraction data support this conclusion: at 180° C. (see FIGS. 8A-8C) and 150° C. (see FIGS. 9A and 9B), the d₅₀ of the Cu(POA)₂ particles is 3.13±0.08 μm and 2.86±0.01 μm or 1.48±0.02 μm (depending on N2 flow see below), respectively. Precursor concentration has no impact on the geometric size of the MOF particles (see FIGS. 8A-8C) but is inversely proportional to the diameter of the MOF nanorods comprising the resulting micrometric spheres: approximately 500 nm for low (1.0 mg/mL POA), 270 nm for medium (1.5 mg/mL POA), and 150 nm for high concentration (3.0 mg/mL POA). See FIG. 10. Smaller monodispersed aerosol particles (1.19±0.28 μm in diameter via SEM; 1.48±0.01 μm with span of 1.20±0.07 via laser diffraction), which lead to more suitable particles for inhalation as discussed later, were achieved by using higher N2 atomizing flow compared to more broadly dispersed, larger particles of 2.52±1.37 μm (SEM) or 2.86±0.01 μm (span 1.31±0.12; laser diffraction) in diameter for lower N2 flow (293 L/h and 15 kPa pressure drop vs 1052 L/h and 75 kPa pressure drop) as shown in FIG. 11. See also laser diffraction data in FIGS. 9A and 9B. Soaking and sonicating the MOF dry powder spheres in abundant water leads to their complete dissolution, which prevents any bioaccumulation assuring the effective release of the Cu(POA)₂ μmolecular species (prodrug). Conversely, when soaked in alcohol, the microparticles are dissembled into their nanoparticle constituents. See FIG. 12. In summary, the following spray drying conditions were determined to lead to the optimal aerosol particles: 1:2 Cu:POA ratio, 80%:20% H₂O:EtOH solvent mixture, 1.5 mg/mL POA precursor concentration, 150° C. inlet temperature, and 1052 L/h N2 flow (75 kPa pressure drop). See FIGS. 2A-2C.

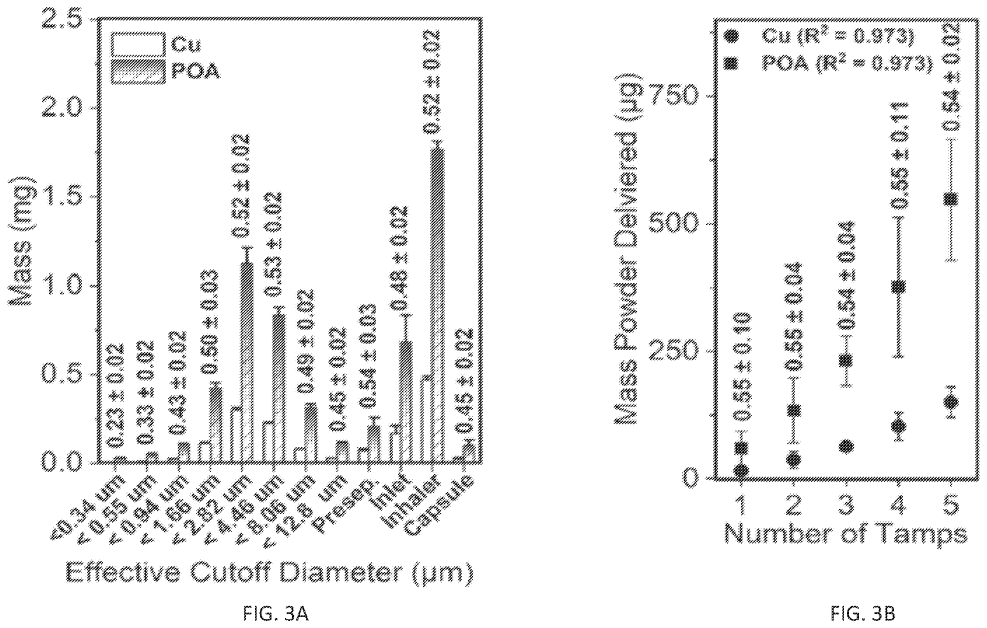

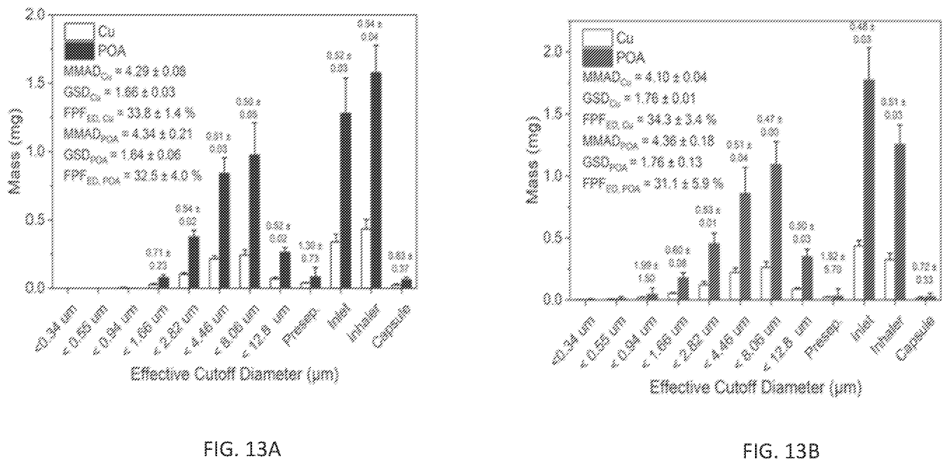

Particle sizing via microscopy or diffraction is not always indicative of the resulting aerosol performance.[22,30] Aerodynamic sizing experiments, specifically mass based inertial impaction, provide more meaningful and discrete characterization data.[22,28] For example, Cu(POA)₂ μmicroparticles obtained at 150° C. and low N2 atomization flow exhibiting 2.52±1.37 μm or 2.86±0.01 μm (span 1.31±0.12) volume diameter (captured via SEM or laser diffraction, respectively) had MMAD>4 μm (see FIGS. 13A and 13B) with a fine particle fraction of the emitted inhaler dose (FPFED)<35% (note inlet temperature had negligible impact on aerodynamic particle sizes). The MMAD was lowered while maintaining crystal structure by increasing the N2 atomizing flow in the spray dryer (decreasing droplet size). Homogeneity in the microparticles was observed as MMAD and geometric standard deviation (GSD) generated via Cu or POA mass analysis were comparable: 2.59±0.02 μm (GSD=1.64±0.02) or 2.55±0.02 μm (GSD=1.69±0.02), respectively. See FIG. 3A. Moreover, the molar ratio of Cu:POA is constant at about 0.5 (as expected with 1:2 Cu:POA precursor concentration) at all analysis points during impaction except for the capsule and final two stages of the impactor where low product collection may have distorted assay results. The FPFED for this optimal synthesis was also roughly to 66.1±2.1% (based on Cu) and 66.0±1.2% (based on POA) which is due to the smaller MMAD. This implies more of the powder would be able to reach the lower airways and is supported by a large decrease in inlet deposition (representative of a throat, see FIGS. 13A and 13B for comparison). FIG. 3B shows dry powder delivery data utilizing custom dosators. [5,33] See FIG. 11. These devices are designed to deliver powder masses <1 mg to mice in vivo and frequently used to assess the disposition of efficacy of anti-TB drugs.[34] Here, a linear relationship between the number of device tamps into a Cu(POA)₂ powder bed and the mass of powder delivered in vitro was generated. The linearity and molar ratio of Cu:POA remained constant regardless of assay method (Cu vs POA). The reproducibility of this method is supported by identical R² values to the literature.[33]

As most pharmaceutical drugs exhibit coordinating functionalities (such as carboxylates, N-heterocycles, phosphonates, etc.) there is wide range of possible MOF (or coordination polymer) formulations that can be attained upon combination with metal cations in aqueous solution. This gives rise to the possibility of preparing inhalable therapies containing metal-drug pairs for other acute pulmonary disorders stemming from bacterial or viral infections in addition to more chronic ailments, such as cancer.

Conclusion: Inhalable MOF aerosol compounds solely of two or three active pharmaceutical ingredients: Cu and POA or Cu, Gd, and POA, have been developed via spray drying as a therapy for treating TB by pulmonary administration. Suitable aerodynamic diameters have been validated by inertial impaction. Spray drying presents an efficient formulation route for inhalable MOF drug aerosols. This approach can mitigate the risk of possible toxic responses due to the presence of inactive species while achieving higher doses per particle without additional excipient.

### REFERENCES

All references listed in the instant disclosure, including but not limited to all patents, patent applications and publications thereof, scientific journal articles, and database entries, are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
1. World Health Organization, “Tuberculosis Fact Sheet”, Mar. 24, 2020.
2. D. J. Sloan, G. R. Davies and S. H. Khoo, Current respiratory medicine reviews, 2013, 9, 200-210.
3. A. J. Hickey, P. G. Durham, A. Dharmadhikari and E. A. Nardell, J Controlled Release, 2016, 240, 127-134.
4. M. Braunstein, A. J. Hickey and S. Ekins, Pharm. Res., 2019, 36, 166; A. Misra, A. J. Hickey, C. Rossi, G. Borchard, H. Terada, K. Makino, P. B. Fourie and P. Colombo, Tuberculosis, 2011, 91, 71-81;
5. I. E. Stewart, P. B. Lukka, J. Liu, B. Meibohm, M. Gonzalez-Juarrero, M. S. Braunstein, R. E. Lee and A. J. Hickey, Pharm. Res., 2019, 36, 136.
6. A. Aziz, M. Ishaq, N. A. Jaffer, R. Akhwand and A. H. Bhatti, Am. Rev. Respir. Dis., 1986, 134, 1056-1061; Y. Zhang, W. Shi, W. Zhang and D. Mitchison, Microbiol Spectr, 2014, 2, Mgm2-0023-2013.
7. Y. Zhang, W. Shi, W. Zhang and D. Mitchison, Microbiology Spectrum, 2014, 2; B. Prideaux, L. E. Via, M. D. Zimmerman, S. Eum, J. Sarathy, P. O'Brien, C. Chen, F. Kaya, D. M. Weiner, P.-Y. Chen, T. Song, M. Lee, T. S. Shim, J. S. Cho, W. Kim, S. N. Cho, K. N. Olivier, C. E. Barry and V. Dartois, Nat. Med. (N. Y., NY, U. S.), 2015, 21, 1223-1227; W. Shi, X. Zhang, X. Jiang, H. Yuan, J. S. Lee, C. E. Barry, 3rd, H. Wang, W. Zhang and Y. Zhang, Science, 2011, 333, 1630-1632.
8. S. Zhang, J. Chen, W. Shi, W. Liu, W. Zhang and Y. Zhang, Emerging Microbes & Infections, 2013, 2, 1-5; Y. Zhang, S. Permar and Z. Sun, J. Med. Microbiol., 2002, 51, 42-49.
9. M. G. Whitfield, H. M. Soeters, R. M. Warren, T. York, S. L. Sampson, E. M. Streicher, P. D. van Helden and A. van Rie, PLoS One, 2015, 10, e0133869; A. Scorpio and Y. Zhang, Nat. Med. (N. Y., NY, U. S.), 1996, 2, 662-667.
10. K. C. Chang, W. W. Yew and Y. Zhang, Antimicrob. Agents Chemother., 2011, 55, 4499-4505; P. Blasi, A. Schoubben, S. Giovagnoli, C. Rossi and M. Ricci, Expert Opinion on Drug Delivery, 2009, 6, 977-993.
11. D. Pires, E. Valente, M. F. Simões, N. Carmo, B. Testa, L. Constantino and E. Anes, Antimicrob. Agents Chemother., 2015, 59, 7693-7699; M. F. Simões, E. Valente, M. J. R. Gómez, E. Anes and L. Constantino, Eur. J. Pharm. Sci., 2009, 37, 257-263.
12. E. F. Young, E. Perkowski, S. Malik, J. D. Hayden, P. G. Durham, L. Zhong, J. T. Welch, M. S. Braunstein and A. J. Hickey, Pharm. Res., 2016, 33, 2495-2505.
13. P. G. Durham, Y. Zhang, N. German, N. Mortensen, J. Dhillon, D. A. Mitchison, P. B. Fourie and A. J. Hickey, Mol. Pharm., 2015, 12, 2574-2581.
14. S. A. Montgomery, E. F. Young, P. G. Durham, K. E. Zulauf, L. Rank, B. K. Miller, J. D. Hayden, F.-C. Lin, J. T. Welch, A. J. Hickey and M. Braunstein, PLoS One, 2018, 13, e0204495; P. G. Durham, E. F. Young, M. S. Braunstein, J. T. Welch and A. J. Hickey, Int. J Pharm., 2016, 514, 384-391.
15. P. Horcajada, T. Chalati, C. Serre, B. Gillet, C. Sebrie, T. Baati, J. F. Eubank, D. Heurtaux, P. Clayette, C. Kreuz, J.-S. Chang, Y. K. Hwang, V. Marsaud, P.-N. Bories, L. Cynober, S. Gil, G. Férey, P. Couvreur and R. Gref, Nat. Mater., 2010, 9, 172-178.
16. W. Cai, J. Wang, C. Chu, W. Chen, C. Wu and G. Liu, Adv. Sci., 2019, 6, 1801526.
17. Y. Chen, P. Li, J. A. Modica, R. J. Drout and O. K. Farha, Journal of the American Chemical Society, 2018, 140, 5678-5681; M. P. Abugafy, B. L. Caetano, B. G. Chiari-Andréo, B. Fonseca-Santos, A. M. do Santos, M. Chorilli and L. A. Chiavacci, European Journal of Pharmaceutics and Biopharmaceutics, 2018, 127, 112-119.
18. H.-X. Zhao, Q. Zou, S.-K. Sun, C. Yu, X. Zhang, R.-J. Li and Y.-Y. Fu, Chemical Science, 2016, 7, 5294-5301; R. Bian, T. Wang, L. Zhang, L. Li and C. Wang, Biomaterials Science, 2015, 3, 1270-1278.
19. X. Hu, C. Wang, L. Wang, Z. Liu, L. Wu, G. Zhang, L. Yu, X. Ren, P. York, L. Sun, J. Zhang and H. Li, International journal of pharmaceutics, 2019, 564, 153-161; G. Wyszogrodzka-Gaweł, P. Dorożyński, S. Giovagnoli, W. Strzempek, E. Pesta, W. P. Wȩglarz, B. Gil, E. Menaszek and P. Kulinowski, Pharmaceutics, 2019, 11; C. Fernández-Paz, S. Rojas, P. Salcedo-Abraira, T. Simón-Yarza, C. Remuñán-López and P. Horcajada, ACS Appl. Mater. Interfaces, 2020.
20. A. Guo, M. Durymanov, A. Permyakova, S. Sene, C. Serre and J. Reineke, Pharm. Res., 2019, 36, 53.
21. B. W. Jhun and W. J. Koh, Tuberculosis and respiratory diseases, 2020, 83, 20-30; M. Gegia, N. Winters, A. Benedetti, D. van Soolingen and D. Menzies, The Lancet Infectious Diseases, 2017, 17, 223-234.
22. A. J. Hickey and D. A. Edwards, J. Pharm. Sci., 2018, 107, 794-796.
23. A. Speer, T. B. Shrestha, S. H. Bossmann, R. J. Basaraba, G. J. Harber, S. M. Michalek, M. Niederweis, O. Kutsch and F. Wolschendorf, Antimicrobial Agents and Chemotherapy, 2013, 57, 1089-1091.
24. R. A. Festa, M. E. Helsel, K. J. Franz and D. J. Thiele, Chemistry & biology, 2014, 21, 977-987; F. Wolschendorf, D. Ackart, T. B. Shrestha, L. Hascall-Dove, S. Nolan, G. Lamichhane, Y. Wang, S. H. Bossmann, R. J. Basaraba and M. Niederweis, Proceedings of the National Academy of Sciences of the United States of America, 2011, 108, 1621-1626; O. Neyrolles, F. Wolschendorf, A. Mitra and M. Niederweis, Immunological reviews, 2015, 264, 249-263.
25. L. E. Via, R. Savic, D. M. Weiner, M. D. Zimmerman, B. Prideaux, S. M. Irwin, E. Lyon, P. O'Brien, P. Gopal, S. Eum, M. Lee, J. P. Lanoix, N. K. Dutta, T. Shim, J. S. Cho, W. Kim, P. C. Karakousis, A. Lenaerts, E. Nuermberger, C. E. Barry, 3rd and V. Dartois, ACS infectious diseases, 2015, 1, 203-214.
26. C. L. Klein, R. J. Majeste, L. M. Trefonas and C. J. O'Connor, Inorg. Chem., 1982, 21, 1891-1897.
27. R. Vehring, Pharm. Res., 2008, 25, 999-1022; D. A. Edwards, J. Hanes, G. Caponetti, J. Hrkach, A. Ben-Jebria, M. L. Eskew, J. Mintzes, D. Deaver, N. Lotan and R. Langer, Science, 1997, 276, 1868-1871; A. J. Hickey, KONA Powder and Particle Journal, 2018, 35, 3-13.
28. A. Hickey and S. da Rocha, Pharmaceutical Inhalation Aerosol Technology, Third Edition., Boca Raton: CRC Press, 2019.
29. N. R. Labiris and M. B. Dolovich, Br. J. Clin. Pharmacol., 2003, 56, 588-599; J. S. Patton and P. R. Byron, Nat. Rev. Drug Discovery, 2007, 6, 67-74.
30. D. A. Edwards, J. Hanes, G. Caponetti, J. Hrkach, A. Ben-Jebria, M. L. Eskew, J. Mintzes, D. Deaver, N. Lotan and R. Langer, Science, 1997, 276, 1868-1872.
31. J. A. Champion, A. Walker and S. Mitragotri, Pharm. Res., 2008, 25, 1815-1821; M. González-Juarrero and M. P. O'Sullivan, Tuberculosis (Edinb), 2011, 91, 86-92; S. Rudt and R. H. Müller, Eur. J. Pharm. Sci., 1993, 1, 31-39.
32. L. Garzón-Tovar, M. Cano-Sarabia, A. Carné-Sánchez, C. Carbonell, I. Imaz and D. Maspoch, Reaction Chemistry & Engineering, 2016, 1, 533-539; A. Came-Sanchez, I. Imaz, M. Cano-Sarabia and D. Maspoch, Nature Chem., 2013, 5, 203-211; J. Troyano, C. Çamur, L. Garzón-Tovar, A. Carné-Sánchez, I. Imaz and D. Maspoch, Acc. Chem. Res., 2020.
33. A. J. Hickey, I. E. Stewart and A. Jones, in Drug Delivery to the Lungs, 2019, vol. 30.
34. D. N. Price, N. K. Kunda, and P. Muttil, KONA Powder and Particle Journal, 2019, 36, 129-144.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A spray-dried metal-organic framework (MOF) particle for treating and/or detecting a pulmonary disease or disorder, the spray-dried MOF particle comprising:
a metal ion, optionally wherein the metal ion is an ion of an element selected from an alkaline metal, an alkaline earth metal, a transition metal, and a lanthanide; and
an organic ligand coordinated to said metal ion, wherein said organic ligand comprises at least two metal coordination sites and wherein the organic ligand has a biological activity related to treatment of the pulmonary disease or disorder, is a prodrug of a therapeutic agent that has a biological activity related to treatment of the pulmonary disease or disorder, and/or has a property useful for biomedical imaging.

2. The spray-dried MOF particle of claim 1, wherein the metal ion has a biological activity related to treatment of the pulmonary disease or disorder and/or a property useful for biomedical imaging, optionally wherein the metal ion is an ion of a transition metal or a lanthanide.

3. The spray-dried MOF particle of claim 2, wherein the metal ion is an ion of an element selected from the group consisting of copper (Cu), iron (Fe), palladium (Pd), zinc (Zn), silver (Ag), gold (Au), manganese (Mn), cobalt (Co), rhodium (Rh), nickel (Ni), tantalum (Ta), titanium (Ti), tungsten (W), yttrium (Y), vanadium (V), platinum (Pt), gadolinium (Gd), and ytterbium (Yb).

4. The spray-dried MOF particle of any one of claims 1-3, wherein the organic ligand is a therapeutic agent selected from an anti-asthmatic, an antihistamine, a antitussive, a bronchodilator, a decongestant, an expectorant, a leukotriene modifier, a lung surfactant, an anti-infective, a corticosteroid, a mast cell stabilizer, a mucolytic, a selective phosphodiesterase-4 inhibitor, or a combination or prodrug thereof.

5. The spray-dried MOF particle of any one of claims 1-4, wherein the spray-dried MOF particle has an average particle diameter of between about 1 μm and about m, optionally between about 2 μm and about 3 μm.

6. The spray-dried MOF particle of any one of claims 1-5, wherein the spray-dried MOF particle is hollow.

7. The spray-dried MOF particle of any one of claims 1-6, wherein the organic ligand comprises pyrazinoic acid.

8. The spray-dried MOF particle of any one of claims 1-7, wherein the spray-dried MOF particle comprises bis(pyrazine-2-carboxylato)copper(II) (Cu(POA)₂), optionally wherein said Cu(POA)₂ is hydrated or solvated.

9. The spray-dried MOF particle of any one of claims 1-7, wherein the spray-dried MOF particle comprises at least two different metal ions.

10. The spray-dried MOF particle of any one of claims 1-7, wherein the spray-dried MOF particle comprises at least two different organic ligands, optionally wherein each of the at least two different organic ligands are selected from the group consisting of an anti-asthmatic, an antihistamine, a antitussive, a bronchodilator, a decongestant, an expectorant, a leukotriene modifier, a lung surfactant, an anti-infective, a corticosteroid, a mast cell stabilizer, a mucolytic, a selective phosphodiesterase-4 inhibitor, and a combination thereof.

11. A therapeutic and/or diagnostic composition, the composition comprising an aerosolized form of a spray-dried MOF particle of claim 1 with an aerodynamic particle size distribution (APSD) in a range suitable for pulmonary delivery, optionally about 1-5 microns.

12. The therapeutic and/or diagnostic composition of claim 11, further comprising an excipient or carrier selected from the group consisting of a sugar, a peptide, a lipid and a surfactant.

13. The therapeutic and/or diagnostic composition of claim 11 or claim 12, wherein the spray-dried MOF comprises at least one metal ion of an element selected from a transition metal and a lanthanide, optionally at least one metal ion of an element selected from copper (Cu), iron (Fe), palladium (Pd), zinc (Zn), silver (Ag), gold (Au), manganese (Mn), cobalt (Co), rhodium (Rh), nickel (Ni), tantalum (Ta), titanium (Ti), tungsten (W), yttrium (Y), vanadium (V), platinum (Pt), gadolinium (Gd), and ytterbium (Yb).

14. The therapeutic and/or diagnostic composition of any one of claims 11-13, wherein the spray-dried MOF comprises at least one ligand comprising at least two metal coordination sites and selected from the group consisting of an anti-asthmatic, an antihistamine, a antitussive, a bronchodilator, a decongestant, an expectorant, a leukotriene modifier, a lung surfactant, an anti-infective, a corticosteroid, a mast cell stabilizer, a mucolytic, a selective phosphodiesterase-4 inhibitor, and a combination thereof.

15. The therapeutic and/or diagnostic composition of any one of claims 11-14, wherein the composition is configured to treat and/or diagnose a pulmonary disease or disorder when administered by inhalation, wherein the pulmonary disease or disorder is selected from a bacterial infection, a viral infection, asthma, chronic obstructive pulmonary disorder (COPD), cystic fibrosis, emphysema, bronchitis, pneumonia, lung cancer and any other disease or disorder treatable using an anti-asthmatic, an antihistamine, an antitussive, a bronchodilator, a decongestant, an expectorant, a leukotriene modifier, a lung surfactant, a corticosteroid, a mast cell stabilizer, a mucolytic, or a selective phosphodiesterase-4 inhibitor.

16. The therapeutic and/or diagnostic composition of claim 15, wherein the therapeutic and/or diagnostic composition is configured to treat tuberculosis (TB) when administered by inhalation.

17. The therapeutic and/or diagnostic composition of any one of claims 11-16, wherein the spray-dried MOF particle comprises bis(pyrazine-2-carboxylato)copper(II) (Cu(POA)₂).

18. The therapeutic and/or diagnostic composition of any one of claims 11-17, wherein the aerosolized formulation of the spray-dried MOF particle comprises an APSD of about 2-3 microns (m).

19. The therapeutic and/or diagnostic composition of any one of claims 11-18, wherein the composition comprises a sugar excipient or carrier, wherein the sugar is selected from maltodextrin, mannitol, trehalose, lactose, and/or dextrose.

20. The therapeutic and/or diagnostic composition of any one of claims 11-19, wherein the composition comprises a peptide excipient or carrier, wherein said peptide is one or more of leucine, lysine, glycine, polyleucine (dileucine, trileucine), and/or polylysine (dilysine).

21. A method of treating a pulmonary disease or disorder in a subject, the method comprising:
providing a subject in need of treatment;
providing an inhalable formulation of a spray-dried MOF particle of claim 1, wherein at least one of the metal ion and the organic ligand has a biological activity related to treatment of the pulmonary disease or disorder; and
administering an effective amount of the inhalable formulation of the spray-dried MOF particle to the subject by inhalation,
wherein the pulmonary disease or disorder in the subject is treated.

22. The method of claim 21, wherein the inhalable formulation of the spray-dried MOF particle comprises an aerosolized form of the spray-dried MOF particle having an aerodynamic particle size distribution (APSD) in a range suitable for pulmonary delivery, optionally 1-5 μm, further optionally 2-3 μm.

23. The method of claim 21 or claim 22, wherein the spray-dried MOF particle is a hollow spray-dried MOF microparticle.

24. The method of any one of claims 21-23, wherein the subject is a human subject.

25. The method of any one of claims 21-24, wherein the spray-dried MOF comprises a metal ion of an element selected from the group consisting of copper (Cu), iron (Fe), palladium (Pd), zinc (Zn), silver (Ag), gold (Au), manganese (Mn), cobalt (Co), rhodium (Rh), nickel (Ni), titanium (Ti), vanadium (V), and platinum (Pt).

26. The method of any one of claims 21-25, wherein the spray-dried MOF comprises an organic ligand having at least two metal coordination sites and which is selected from the group consisting of an anti-asthmatic, an antihistamine, an antitussive, a bronchodilator, a decongestant, an expectorant, a leukotriene modifier, a lung surfactant, an anti-infective, a corticosteroid, a mast cell stabilizer, a mucolytic, a selective phosphodiesterase-4 inhibitor, and a combination or prodrug thereof.

27. The method of any one of claims 21-26, wherein the subject has a pulmonary disease or disorder selected from a bacterial infection, a viral infection, asthma, chronic obstructive pulmonary disorder (COPD), cystic fibrosis, emphysema, bronchitis, pneumonia, lung cancer, and any other disease or disorder treatable using an anti-asthmatic, an antihistamine, an antitussive, a bronchodilator, a decongestant, an expectorant, a leukotriene modifier, a lung surfactant, a corticosteroid, a mast cell stabilizer, a mucolytic, or a selective phosphodiesterase-4 inhibitor.

28. The method of claim 27, wherein the subject has tuberculosis (TB).

29. The method of claim 28, wherein the TB in the subject comprises multiple drug resistant (MDR) TB or extensively drug resistant (XDR) TB.

30. The method of any one of claims 21-29, wherein the spray-dried MOF particle comprises bis(pyrazine-2-carboxylato)copper(II) (Cu(POA)₂).

31. The method of claim 30, wherein the spray-dried MOF particle further comprises a metal ion selected from Fe, Pd, Zn and Gd.

32. The method of any one of claims 21-31, wherein the subject is also treated with a second therapeutic composition for treating the pulmonary disease or disorder, wherein the second therapeutic composition is administered orally or intravenously.

33. A method of diagnosing a pulmonary disease or disorder in a subject, the method comprising:
providing a subject suspected or at risk of having a pulmonary disease or disorder;
providing an inhalable formulation of a spray-dried MOF particle of claim 1, wherein the spray-dried MOF comprises at least one metal ion or at least one organic ligand having a property useful in biomedical imaging;
administering an effective amount of the inhalable formulation of the spray-dried MOF particle to the subject by inhalation;
imaging at least a portion of at least one lung of the subject; and
analyzing results of the imaging and finding evidence of the presence of a pulmonary disease or disorder; thereby diagnosing the pulmonary disease or disorder in the subject.

34. The method of claim 33, wherein the spray-dried MOF particle comprises a metal ion of an element selected from Gd, Fe, Mn, Au, Y, Yb, Ni, Cu, W, Ta, Pt, and Ti, optionally wherein the metal ion is an Gd ion, a Fe ion, or a Mn ion.

35. The method of claim 33 or claim 34, wherein the imaging comprises performing magnetic resonance imaging (MRI) or computed tomography (CT).
